SEQUENCE LISTING (13 sequences)

SEQ ID NO: 1 - protein, 1213 aa, Artificial Sequence
  SEQ ID NO 1- Exemplary CFH Amino Acid Sequence
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 1231 aa, Artificial Sequence
  SEQ ID NO 2- Exemplary Precursor CFH Amino Acid Sequence
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 3 - protein, 1231 aa, Artificial Sequence
  SEQ ID NO 3- Exemplary Precursor CFH Amino Acid Sequence
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 4 - protein, 449 aa, Artificial Sequence
  SEQ ID NO 4- FHL1 Amino Acid Sequence
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 5 - protein, 246 aa, Artificial Sequence
  SEQ ID NO 5- CFH (1-4) Amino Acid Sequence
DCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEK

SEQ ID NO: 6 - protein, 304 aa, Artificial Sequence
  SEQ ID NO 6- CFH (1-5) Amino Acid Sequence
DCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 7 - protein, 426 aa, Artificial Sequence
  SEQ ID NO 7- CFH (1-7) Amino Acid Sequence
DCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRV

SEQ ID NO: 8 - protein, 126 aa, Artificial Sequence
  SEQ ID NO 8- CFH (19-20) Amino Acid Sequence
TGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 9 - protein, 185 aa, Artificial Sequence
  SEQ ID NO 9- CFH (18-20) Amino Acid Sequence
SCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 10 - DNA, 3696 bp, Artificial Sequence
  SEQ ID NO 10- Exemplary CFH Nucleotide Sequence
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 11 - DNA, 1381 bp, Artificial Sequence
  SEQ ID NO 11-Exemplary Codon Optimized Human Factor H Like 1 (FHL1)
gcggccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctaataagcttggatccagatct

SEQ ID NO: 12 - DNA, 1381 bp, Artificial Sequence
  SEQ ID NO 12-Exemplary Codon Optimized Human Factor H Like 1 (FHL1)
gcggccgccaccatgagactgctggctaaaattatctgcctgatgctgtgggctatctgcgtcgctgaggattgtaacgagctgcccccccggagaaatacagagatcctgaccggctcttggagcgaccagacatatcccgagggcacccaggccatctacaagtgcaggcctggctatcgctctctgggcaacgtgatcatggtgtgcaggaagggagagtgggtggccctgaatcccctgaggaagtgccagaagcgcccttgtggacacccaggcgacacacccttcggcacctttacactgaccggcggcaacgtgttcgagtacggcgtgaaggccgtgtatacctgcaacgagggctaccagctgctgggcgagatcaattacagagagtgtgacacagatggctggaccaacgatatccctatctgcgaggtggtgaagtgtctgcctgtgaccgccccagagaatggcaagatcgtgagctccgccatggagccagacagggagtatcacttcggccaggccgtgcgcttcgtgtgcaactccggctacaagatcgagggcgatgaggagatgcactgtagcgacgatggcttctggtccaaggagaagcccaagtgcgtggagatcagctgtaagtcccctgacgtgatcaatggctctccaatcagccagaagatcatctataaggagaacgagaggtttcagtacaagtgcaatatgggctacgagtattctgagaggggcgatgccgtgtgcacagagagcggatggcggcccctgccttcctgcgaggagaagtcttgtgacaacccttatatcccaaatggcgattacagcccactgcggatcaagcacagaacaggcgatgagatcacctatcagtgccggaacggcttttaccccgccacaagaggcaataccgccaagtgtacatccaccggatggatcccagcaccaagatgcaccctgaagccctgtgactatcctgatatcaagcacggcggcctgtatcacgagaacatgagacggccctacttccctgtggccgtgggcaagtactattcctactattgcgacgagcactttgagacaccctccggctcttactgggaccacatccactgtacccaggatggatggagccccgcagtgccatgcctgaggaagtgttacttcccttatctggagaatggctacaaccagaattatggccgcaagtttgtgcagggcaagagcatcgatgtggcatgccacccaggatacgcactgccaaaggcacagaccacagtgacctgtatggaaaacggctggtcccctacccctcgctgtatcagagtgtcattcaccctgtaataagcttggatccagatct

SEQ ID NO: 13 - DNA, 1381 bp, Artificial Sequence
  SEQ ID NO 13-Exemplary Non-Codon Optimized Human Factor H Like 1 (FHL1)
gcggccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatcgaagtcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctaataagcttggatccagatct

(19) United States
(12) Patent Application Publication
(10) Pub. No.: US 2022/0395557 A1
(43) Pub. Date: Dec. 15, 2022

(54) METHODS FOR TREATING PATIENTS HAVING CFH MUTATIONS WITH RECOMBINANT CFH PROTEINS

(71) Applicant: Gemini Therapeutics Sub, Inc., Cambridge, MA (US)

(72) Inventors: James McLaughlin, Cambridge, MA (US); Lisa Huang, Cambridge, MA (US); Scott Lauder, Andover, MA (US); Suresh Katti, Hamden, CT (US)

(21) Appl. No.: 17/771,334

(22) PCT Filed: Oct. 23, 2020
(86) PCT No.: PCT/US2020/057155

§ 371 Date: Apr. 22, 2022

Related U.S. Application Data
(60) Provisional application No. 62/925,071, filed on Oct. 23, 2019.

(51) Int. Cl. A61K 38/17; C07K 14/47; C07K 16/22

(52) U.S. Cl. CPC A61K 38/1725; C07K 14/472; C07K 16/22; C07K 2317/76; C12Q 1/6883

(57) ABSTRACT

The present disclosure provides methods for treating, preventing, or inhibiting diseases in patients having one or more mutations in complement factor H (CFH), complement component 3 (C3), and complement factor B (CFB). Also provided are combination therapies comprising a CFH protein and a VEGF antagonist for treating neovascularization-associated ocular diseases.

### CROSS-REFERENCE TO RELATED APPLICATIONS

This application claims the benefit of and priority to U.S. Provisional Patent Application No. 62/925,071, filed Oct. 23, 2019, the disclosures of which are incorporated by reference herein in their entireties for all purposes.

### SEQUENCE LISTING

The instant application contains a Sequence Listing which has been submitted electronically in ASCII format and is hereby incorporated by reference in its entirety. Said ASCII copy, created on Oct. 22, 2020, is named GEM-015WO_SL_ST25.txt and is 58,992 bytes in size.

### BACKGROUND OF THE DISCLOSURE

Age-related macular degeneration (AMD) is a medical condition and is the leading cause of legal blindness in Western societies. AMD typically affects older adults and results in a loss of central vision due to degenerative and neovascular changes to the macula, a pigmented region at the center of the retina which is responsible for visual acuity. There are four major AMD subtypes: Early AMD; Intermediate AMD; Advanced non-neovascular (“Dry”) AMD; and Advanced neovascular (“Wet”) AMD. Typically, AMD is identified by the focal hyperpigmentation of the retinal pigment epithelium (RPE) and accumulation of drusen deposits and/or geographic atrophy. The size and number of drusen deposits and level of geographic atrophy typically correlates with AMD severity.

AMD occurs in up to 8% of individuals over the age of 60, and the prevalence of AMD continues to increase with age. The U.S. is anticipated to have nearly 22 million cases of AMD by the year 2050, while global cases of AMD are expected to be nearly 288 million by the year 2040.

There is a need for novel treatments for preventing progression from early to intermediate and/or from intermediate to advanced stages of AMD to prevent loss of vision, particularly in certain subpopulations of patients having one or more CFH mutations.

### SUMMARY OF THE DISCLOSURE

The present disclosure provides methods for treating, preventing, or inhibiting a disease or disorder in a subject (e.g., a subject having a mutation in certain complement pathway genes) by administering an effective amount of a recombinant CFH proteins or biologically active fragments and/or variants thereof. The disease can be a disease of the eye, and the recombinant CFH proteins or biologically active fragments and/or variants thereof can be administered intraocularly (e.g., intravitreally).

In one aspect, the present disclosure provides a method of treating a subject having a disease or disorder associated with undesired activity of the alternative complement pathway, comprising the step of administering to the subject a complement factor H (CFH) polypeptide or biologically active fragment and/or variant thereof, wherein the subject has one or more CFH, complement component 3 (C3), and/or complement factor B (CFB) gene mutations.

In another aspect, the present disclosure provides a method of treating a subject having age-related macular degeneration (AMD), comprising the step of administering to the subject a complement factor H (CFH) polypeptide or biologically active fragment and/or variant thereof, wherein the subject has one or more CFH, C3, and/or CFB gene mutations.

In another aspect, the present disclosure provides a method of treating a subject having an ocular disease associated with neovascularization, the method comprising the steps of: (a) administering to the subject a complement factor H (CFH) polypeptide or biologically active fragment and/or variant thereof; and (b) administering to the subject a VEGF antagonist. In certain embodiments, the subject has one or more CFH, C3, and/or CFB gene mutations.

In certain embodiments, the VEGF antagonist comprises an antibody or antigen-binding fragment thereof that binds VEGF. In certain embodiments, the antibody or antigen-binding fragment thereof is selected from the group consisting of ranibizumab and bevacizumab. In certain embodiments, the VEGF antagonist is aflibercept. In certain embodiments, the ocular disease associated with neovascularization is neovascular AMD. In certain embodiments, the ocular disease associated with neovascularization is diabetic retinopathy (e.g., diabetic macular edema).

In certain embodiments, the CFH polypeptide or biologically active fragment and/or variant thereof is capable of acting as a cofactor with CFI to facilitate C3b cleavage. In certain embodiments, the CFH polypeptide or biologically active fragment and/or variant thereof is capable of diffusing across the Bruch's membrane. In certain embodiments, the CFH polypeptide or biologically active fragment and/or variant thereof is capable of binding C3b. In certain embodiments, the CFH polypeptide or biologically active fragment and/or variant thereof is capable of facilitating the breakdown of C3b. In certain embodiments, the CFH polypeptide or biologically active fragment and/or variant thereof is capable of binding to a cell surface. In certain embodiments, the CFH polypeptide or biologically active fragment and/or variant thereof is capable of binding to heparin. In certain embodiments, the CFH polypeptide or biologically active fragment and/or variant thereof is capable of reducing C5b9 levels generated as a result of complement activation. In certain embodiments, the CFH polypeptide or biologically active fragment and/or variant thereof is capable of inhibiting hemolysis.

In certain embodiments, the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 3, or a biologically active fragment thereof. In certain embodiments, the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 1, or a biologically active fragment thereof. In certain embodiments, the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 2, or a biologically active fragment thereof. In certain embodiments, the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 4, or a biologically active fragment thereof. In certain embodiments, the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 5, or a biologically active fragment thereof. In certain embodiments, the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 6, or a biologically active fragment thereof. In certain embodiments, the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 7, or a biologically active fragment thereof. In certain embodiments, the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 8, or a biologically active fragment thereof. In certain embodiments, the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 9, or a biologically active fragment thereof. In certain embodiments, the CFH polypeptide or biologically active fragment and/or variant thereof is encoded by a nucleotide sequence that is at least 70%, 75%, 80%, 85%, 90%, 91%, 92%, 93%, 94%, 95%, 96%, 97%, 98%, 99% or 100% identical to the nucleotide sequence of any one of SEQ ID NOs: 10-13, or a biologically active fragment thereof.

In certain embodiments, the CFH polypeptide or biologically active fragment and/or variant thereof comprises the V62 polymorphism. In certain embodiments, the CFH polypeptide or biologically active fragment and/or variant thereof comprises the Y402 polymorphism.

In certain embodiments, the CFH polypeptide or biologically active fragment and/or variant thereof is a mature CFH polypeptide or biologically active fragment and/or variant thereof.

In certain embodiments, the subject is a human. In certain embodiments, the human is at least 40 years of age. In certain embodiments, the human is at least 50 years of age. In certain embodiments, the human is at least 65 years of age.

In certain embodiments, the CFH polypeptide or biologically active fragment and/or variant thereof is administered locally. In certain embodiments, the CFH polypeptide or biologically active fragment and/or variant thereof is administered intravitreally. In certain embodiments, the CFH polypeptide or biologically active fragment and/or variant thereof is administered systemically.

In certain embodiments, the subject has a mutation in the subject's CFH gene, optionally wherein the mutation is a loss-of-function mutation. In certain embodiments, the subject has one or more of the following CFH mutations: Y402H, R2T, L3V, R53C, R53H, S58A, D90G, D130N, R175Q, R175P, I221V, R303W, R303Q, Q400K, P503A, R567G, G650V, S890I, T956M, G1194D, and R1210C. In certain embodiments, the subject has one or more of the following CFH mutations: R2T, R53C, R53H, S58A, D130N, R175Q, R175P, I221V, R303W, R303Q, P503A, R567G, G650V, G1194D, and R1210C. In certain embodiments, the subject has a Y402H mutation. In certain embodiments, the subject is homozygous for a Y402H mutation. In certain embodiments, the subject has an R2T mutation. In certain embodiments, the subject has an L3V mutation. In certain embodiments, the subject has an R53C mutation. In certain embodiments, the subject has an R53H mutation. In certain embodiments, the subject has an S58A mutation. In certain embodiments, the subject has a D90G mutation. In certain embodiments, the subject has a D130N mutation. In certain embodiments, the subject has an R175Q mutation. In certain embodiments, the subject has an R175P mutation. In certain embodiments, the subject has an I221V mutation. In certain embodiments, the subject has an R303W mutation. In certain embodiments, the subject has an R303Q mutation. In certain embodiments, the subject has a Q400K mutation. In certain embodiments, the subject has a P503A mutation. In certain embodiments, the subject has an R567G mutation. In certain embodiments, the subject has a G650V mutation. In certain embodiments, the subject has an S890I mutation. In certain embodiments, the subject has a T956M mutation. In certain embodiments, the subject has a G1194D mutation. In certain embodiments, the subject has an R1210C mutation.

In certain embodiments, the subject expresses a mutant CFH polypeptide having reduced CFH activity as compared to a wildtype CFH polypeptide. In certain embodiments, the CFH activity is the ability to bind to C3b. In certain embodiments, the CFH activity has the ability to act as a cofactor with CFI and facilitate C3b cleavage. In certain embodiments, the CFH activity is the ability to bind to a cell surface. In certain embodiments, the CFH activity is the ability to bind to heparin. In certain embodiments, the CFH activity is the ability to reduce C5b9 levels generated as a result of complement activation. In certain embodiments, the CFH activity is the ability to inhibit hemolysis. In certain embodiments, the wildtype CFH polypeptide comprises the amino acid sequence of SEQ ID NO: 1, 2, or 3.

In certain embodiments, the subject has a mutation in the subject's C3 gene, optionally wherein the mutation is a gain-of-function mutation. In certain embodiments, the subject has one or more of the following C3 mutations: R102G, K155Q, V619M, and R735W.

In certain embodiments, the subject has a mutation in the subject's CFB gene, optionally wherein the mutation is a gain-of-function mutation. In certain embodiments, the subject has the I242L mutation of CFB.

In certain embodiments, the subject is homozygous for CFH 62V, C3 102G, and complement factor B (CFB) 32R.

In certain embodiments, the subject is homozygous for at least one of the one or more CFH, C3, and/or CFB mutations. In certain embodiments, the subject is heterozygous for at least one of the one or more CFH, C3, and/or CFB mutations.

In certain embodiments, the subject has been determined to have the one or more CFH, C3, and/or CFB mutations.

In certain embodiments, the subject has atypical hemolytic uremic syndrome (aHUS). In certain embodiments, the subject has a renal disease or complication.

### BRIEF DESCRIPTION OF THE DRAWINGS

FIG. 2A shows a simplified schematic of the CFH protein bound to C3b. CCPs 1-20 are shown, along with the site of the R53C mutation. FIG. 1B is a graph showing CFH binding ratio to C3b protein. FIG. 1C is a graph plotting cofactor activity of CFH protein. FIG. 1D is a graph showing results from a decay acceleration assay. FIG. 1E is a graph showing results from a Weislab activity assay. FIG. 1F is a graph showing results from a hemolysis inhibition assay. In each assay, VYE control CFH was tested as compared to the R53C mutant CFH protein. The VYE control CFH used in these and other experiments described herein included three prevalent polymorphisms: V62, Y402 and E936. As shown in FIGS. 1B-1F, the R53C mutant CFH has impaired function.

FIG. 2A shows a simplified schematic of the CFH protein bound to C3b. CCPs 1-20 are shown, along with the site of the R53H mutation. FIG. 2B is a graph showing CFH binding ratio to C3b protein. FIG. 2C is a graph plotting cofactor activity of CFH protein. FIG. 2D is a graph showing results from a decay acceleration assay. FIG. 2E is a graph showing results from a Weislab activity assay. FIG. 2F is a graph showing results from a hemolysis inhibition assay. In each assay, VYE control CFH was tested as compared to the R53H mutant CFH protein. As shown in FIGS. 2C-2F, the R53H mutant CFH has impaired function.

FIG. 3A shows a simplified schematic of the CFH protein bound to C3b. CCPs 1-20 are shown, along with the site of the S58A mutation. FIG. 3B is a graph showing CFH binding ratio to C3b protein. FIG. 3C is a graph plotting cofactor activity of CFH protein. FIG. 3D is a graph showing results from a decay acceleration assay. FIG. 3E is a graph showing results from a Weislab activity assay. FIG. 3F is a graph showing results from a hemolysis inhibition assay. In each assay, VYE control CFH was tested as compared to the S58A mutant CFH protein. As shown in FIG. 3C, the S58A mutant CFH has impaired function.

FIG. 4A shows a simplified schematic of the CFH protein bound to C3b. CCPs 1-20 are shown, along with the site of the D90G mutation. FIG. 4B is a graph showing CFH binding ratio to C3b protein. FIG. 4C is a graph plotting cofactor activity of CFH protein. FIG. 4D is a graph showing results from a decay acceleration assay. FIG. 4E is a graph showing results from a Weislab activity assay. FIG. 4F is a graph showing results from a hemolysis inhibition assay. In each assay, VYE control CFH was tested as compared to the D90G mutant CFH protein.

FIG. 5A shows a simplified schematic of the CFH protein bound to C3b. CCPs 1-20 are shown, along with the site of the D130N mutation. FIG. 5B is a graph showing CFH binding ratio to C3b protein. FIG. 5C is a graph plotting cofactor activity of CFH protein. FIG. 5D is a graph showing results from a decay acceleration assay. FIG. 5E is a graph showing results from a Weislab activity assay. FIG. 5F is a graph showing results from a hemolysis inhibition assay. In each assay, VYE control CFH was tested as compared to the D130N mutant CFH protein. As shown in FIGS. 5C, 5D and potentially FIGS. 5E and 5F, the D130N mutant CFH has impaired function.

FIG. 6A shows a simplified schematic of the CFH protein bound to C3b. CCPs 1-20 are shown, along with the site of the R175Q mutation. FIG. 6B is a graph showing CFH binding ratio to C3b protein. FIG. 6C is a graph plotting cofactor activity of CFH protein. FIG. 6D is a graph showing results from a decay acceleration assay. FIG. 6E is a graph showing results from a Weislab activity assay. FIG. 6F is a graph showing results from a hemolysis inhibition assay. In each assay, VYE control CFH was tested as compared to the R175Q mutant CFH protein. As shown in FIGS. 6B-6E, the R175Q mutant CFH has impaired function.

FIG. 7A shows a simplified schematic of the CFH protein bound to C3b. CCPs 1-20 are shown, along with the site of the R175P mutation. FIG. 7B is a graph showing CFH binding ratio to C3b protein. FIG. 7C is a graph plotting cofactor activity of CFH protein. FIG. 7D is a graph showing results from a decay acceleration assay. FIG. 7E is a graph showing results from a Weislab activity assay. FIG. 7F is a graph showing results from a hemolysis inhibition assay. In each assay, VYE control CFH was tested as compared to the R175P mutant CFH protein. As shown in FIGS. 7B-7F, the R175P mutant CFH has impaired function.

FIG. 8A shows a simplified schematic of the CFH protein bound to C3b. CCPs 1-20 are shown, along with the site of the I221V mutation. FIG. 8B is a graph showing CFH binding ratio to C3b protein. FIG. 8C is a graph plotting cofactor activity of CFH protein. FIG. 8D is a graph showing results from a decay acceleration assay. FIG. 8E is a graph showing results from a Weislab activity assay. FIG. 8F is a graph showing results from a hemolysis inhibition assay. In each assay, VYE control CFH was tested as compared to the I221V mutant CFH protein. As shown in FIG. 8C and potentially FIG. 8E, the I221V mutant CFH has impaired function.

FIG. 9A shows a simplified schematic of the CFH protein bound to C3b. CCPs 1-20 are shown, along with the site of the R303W mutation. FIG. 9B is a graph showing CFH binding ratio to C3b protein. FIG. 9C is a graph plotting cofactor activity of CFH protein. FIG. 9D is a graph showing results from a decay acceleration assay. FIG. 9E is a graph showing results from a Weislab activity assay. FIG. 9F is a graph showing results from a hemolysis inhibition assay. In each assay, VYE control CFH was tested as compared to the R303W mutant CFH protein. As shown in FIG. 9F and potentially FIGS. 9C and 9E, the R303W mutant CFH has impaired function.

FIG. 10A shows a simplified schematic of the CFH protein bound to C3b. CCPs 1-20 are shown, along with the site of the R303Q mutation. FIG. 10B is a graph showing CFH binding ratio to C3b protein. FIG. 10C is a graph plotting cofactor activity of CFH protein. FIG. 10D is a graph showing results from a decay acceleration assay. FIG. 10E is a graph showing results from a Weislab activity assay. FIG. 10F is a graph showing results from a hemolysis inhibition assay. In each assay, VYE control CFH was tested as compared to the R303Q mutant CFH protein. As shown in FIGS. 10B and 10F and potentially 10C-10E, the R303Q mutant CFH has impaired function.

FIG. 11A shows a simplified schematic of the CFH protein bound to C3b. CCPs 1-20 are shown, along with the site of the Q400K mutation. FIG. 11B is a graph showing CFH binding ratio to C3b protein. FIG. 11C is a graph plotting cofactor activity of CFH protein. FIG. 11D is a graph showing results from a decay acceleration assay. FIG. 11E is a graph showing results from a Weislab activity assay. FIG. 11F is a graph showing results from a hemolysis inhibition assay. In each assay, VYE control CFH was tested as compared to the Q400K mutant CFH protein.

FIG. 12A shows a simplified schematic of the CFH protein bound to C3b. CCPs 1-20 are shown, along with the site of the Y402H mutation. FIG. 12B is a graph showing CFH binding ratio to C3b protein. FIG. 12C is a graph plotting cofactor activity of CFH protein. FIG. 12D is a graph showing results from a decay acceleration assay. FIG. 12E is a graph showing results from a Weislab activity assay. FIG. 12F is a graph showing results from a hemolysis inhibition assay. In each assay, VYE control CFH was tested as compared to the Y402H mutant CFH protein.

FIG. 13A shows a simplified schematic of the CFH protein bound to C3b. CCPs 1-20 are shown, along with the site of the P503A mutation. FIG. 13B is a graph showing CFH binding ratio to C3b protein. FIG. 13C is a graph plotting cofactor activity of CFH protein.

FIG. 13D is a graph showing results from a decay acceleration assay. FIG. 13E is a graph showing results from a Weislab activity assay. FIG. 13F is a graph showing results from a hemolysis inhibition assay. In each assay, VYE control CFH was tested as compared to the P503A mutant CFH protein. As shown in FIGS. 13B and 13E-13F and potentially FIG. 13D, the P503A mutant CFH has impaired function.

FIG. 14A shows a simplified schematic of the CFH protein bound to C3b. CCPs 1-20 are shown, along with the site of the R567G mutation. FIG. 14B is a graph showing CFH binding ratio to C3b protein. FIG. 14C is a graph plotting cofactor activity of CFH protein. FIG. 14D is a graph showing results from a decay acceleration assay. FIG. 14E is a graph showing results from a Weislab activity assay. FIG. 14F is a graph showing results from a hemolysis inhibition assay. In each assay, VYE control CFH was tested as compared to the R567G mutant CFH protein. As shown in FIGS. 14B and 14D-14F and potentially 14C, the R567G mutant CFH has impaired function.

FIG. 15A shows a simplified schematic of the CFH protein bound to C3b. CCPs 1-20 are shown, along with the site of the G650V mutation. FIG. 15B is a graph showing CFH binding ratio to C3b protein. FIG. 15C is a graph plotting cofactor activity of CFH protein. FIG. 15D is a graph showing results from a decay acceleration assay. FIG. 15E is a graph showing results from a Weislab activity assay. FIG. 15F is a graph showing results from a hemolysis inhibition assay. In each assay, VYE control CFH was tested as compared to the G650V mutant CFH protein. As shown in FIG. 15C, the G650V mutant CFH has impaired function.

FIG. 16A shows a simplified schematic of the CFH protein bound to C3b. CCPs 1-20 are shown, along with the site of the S890I mutation. FIG. 16B is a graph showing CFH binding ratio to C3b protein. FIG. 16C is a graph plotting cofactor activity of CFH protein. FIG. 16D is a graph showing results from a decay acceleration assay. FIG. 16E is a graph showing results from a Weislab activity assay. FIG. 16F is a graph showing results from a hemolysis inhibition assay. In each assay, VYE control CFH was tested as compared to the S890I mutant CFH protein.

FIG. 17A shows a simplified schematic of the CFH protein bound to C3b. CCPs 1-20 are shown, along with the site of the T956M mutation. FIG. 17B is a graph showing CFH binding ratio to C3b protein. FIG. 17C is a graph plotting cofactor activity of CFH protein. FIG. 17D is a graph showing results from a decay acceleration assay. FIG. 17E is a graph showing results from a Weislab activity assay. FIG. 17F is a graph showing results from a hemolysis inhibition assay. In each assay, VYE control CFH was tested as compared to the T956M mutant CFH protein.

FIG. 18A shows a simplified schematic of the CFH protein bound to C3b. CCPs 1-20 are shown, along with the site of the G1194D mutation. FIG. 18B is a graph showing CFH binding ratio to C3b protein. FIG. 18C is a graph plotting cofactor activity of CFH protein. FIG. 18D is a graph showing results from a decay acceleration assay. FIG. 18E is a graph showing results from a Weislab activity assay. FIG. 18F is a graph showing results from a hemolysis inhibition assay. In each assay, VYE control CFH was tested as compared to the G1194D mutant CFH protein. As shown in FIGS. 18C-18F, the G1194D mutant CFH has impaired function.

FIG. 19A shows a simplified schematic of the CFH protein bound to C3b. CCPs 1-20 are shown, along with the site of the R1210C mutation. FIG. 19B is a graph showing CFH binding ratio to C3b protein. FIG. 19C is a graph plotting cofactor activity of CFH protein. FIG. 19D is a graph showing results from a decay acceleration assay. FIG. 19E is a graph showing results from a Weislab activity assay. FIG. 19F is a graph showing results from a hemolysis inhibition assay. In each assay, VYE control CFH was tested as compared to the R1210C mutant CFH protein. As shown in FIGS. 19B-19D and potentially 19E and 19F, the R1210C mutant CFH has impaired function.

FIG. 20A shows a simplified schematic of the CFH protein bound to C3b. CCPs 1-20 are shown. FIG. 20B is a graph showing CFH binding ratio to C3b protein. FIG. 20C is a graph plotting cofactor activity of CFH protein. FIG. 20D is a graph showing results from a decay acceleration assay. FIG. 20E is a graph showing results from a Weislab activity assay. FIG. 20F is a graph showing results from a hemolysis inhibition assay. In each assay, VYE control CFH was tested as compared to the R2T mutant CFH protein.

FIG. 21A shows a simplified schematic of the CFH protein bound to C3b. CCPs 1-20 are shown. FIG. 21B is a graph showing CFH binding ratio to C3b protein. FIG. 21C is a graph plotting cofactor activity of CFH protein. FIG. 21D is a graph showing results from a decay acceleration assay. FIG. 21E is a graph showing results from a Weislab activity assay. FIG. 21F is a graph showing results from a hemolysis inhibition assay. In each assay, VYE control CFH was tested as compared to the L3V mutant CFH protein.

FIG. 22 shows a bar graph summarizing the C3b binding assay results from FIGS. 1B, 2B, 3B, 4B, 5B, 6B, 7B, 8B, 9B, 10B, 11B, 12B, 13B, 14B, 15B, 16B, 17B, 18B, 19B, 20B, and 21B based on ratios as compared to the VYE CFH control values. CFH mutants having ratios<0.9 were identified as being functionally deficient in the C3b binding assay. The dotted line corresponds to the 0.9 ratio. Bars above that line are indicated in bars with dots, while bars below that line are indicated in bars with stripes. The black bar corresponds to the VYE control.

FIG. 23 shows a bar graph summarizing the cofactor activity assay results from FIGS. 1C, 2C, 3C, 4C, 5C, 6C, 7C, 8C, 9C, 10C, 11C, 12C, 13C, 14C, 15C, 16C, 17C, 18C, 19C, 20C, and 21C based on ratios as compared to the VYE CFH control values. CFH mutants having ratios>1.1 were identified as being functionally deficient in the cofactor activity assay. The dotted line corresponds to the 1.1 ratio. Bars above the dashed line are indicated in bars with stripes, while bars below that line are indicated in bars with dots. The black bar corresponds to the VYE control.

FIG. 24 shows a bar graph summarizing the decay acceleration assay results from FIGS. 1D, 2D, 3D, 4D, 5D, 6D, 7D, 8D, 9D, 10D, 11D, 12D, 13D, 14D, 15D, 16D, 17D, 18D, 19D, 20D, and 21D based on ratios as compared to the VYE CFH control values. CFH mutants having ratios<0.9 were identified as being functionally deficient in the decay acceleration assay. The dotted line corresponds to the 0.9 ratio. Bars above that line are indicated in bars with dots, while bars below that line are indicated in bars with stripes. The black bar corresponds to the VYE control.

FIG. 25 shows a bar graph summarizing the Weislab assay results from FIGS. 1E, 2E, 3E, 4E, 5E, 6E, 7E, 8E, 9E, 10E, 11E, 12E, 13E, 14E, 15E, 16E, 17E, 18E, 19E, 20E, and 21E based on ratios as compared to the VYE CFH control values. CFH mutants having ratios>1.6 were identified as being functionally deficient in the Weislab® assay. The dotted line corresponds to the 1.6 ratio. Bars above that line are indicated in bars with stripes, while bars below that line are indicated in bars with dots. The black bar corresponds to the VYE control.

FIG. 26 shows a bar graph comparing the cell expression of VYE control CFH as compared to each of the respective CFH mutants. CFH mutants having ratios<0.9 were identified as being associated with functionally deficient with regard to cell expression. The dotted line corresponds to the 0.9 ratio. Bars above that line are indicated in bars with stripes, while bars below that line are indicated in bars with dots. The black bar corresponds to the VYE control.

FIG. 27 is a table indicating whether each CFH mutant was identified as being functionally deficient in each of the recited assays: the cell expression assay, the C3b binding assay, the decay acceleration assay, the cofactor activity assay, the decay acceleration assay, the Weislab® assay and the hemolysis assay. “Y” means functionally deficient in the respective assay, “N” means not functionally deficient in the respective assay, and “M” means maybe functionally deficient in the respective assay.

### DETAILED DESCRIPTION OF THE DISCLOSURE

The present disclosure provides methods for treating, preventing, or inhibiting a disease or disorder in a subject (e.g., a subject having a mutation in certain complement pathway genes) by administering an effective amount of a recombinant CFH proteins or biologically active fragments and/or variants thereof. The disease can be a disease of the eye, and the recombinant CFH proteins or biologically active fragments and/or variants thereof can be administered intraocularly (e.g., intravitreally).

In one aspect, the present disclosure provides a method of treating a subject having a disease or disorder associated with undesired activity of the alternative complement pathway, comprising the step of administering to the subject a complement factor H (CFH) polypeptide or biologically active fragment and/or variant thereof, wherein the subject has one or more CFH, complement component 3 (C3), and/or complement factor B (CFB) gene mutations. In another aspect, the present disclosure provides a method of treating a subject having a disease or disorder associated with undesired activity of the alternative complement pathway, the method comprising the steps of (a) selecting a subject that has one or more CFH, C3, and/or CFB gene mutations; and (b) administering to the subject a complement factor H (CFH) polypeptide or biologically active fragment and/or variant thereof.

In another aspect, the instant disclosure provides a method of treating a subject having age-related macular degeneration (AMD), comprising the step of administering to the subject a complement factor H (CFH) polypeptide or biologically active fragment and/or variant thereof, wherein the subject has one or more CFH, C3, and/or CFB gene mutations. In another aspect, the instant disclosure provides a method of treating a subject having age-related macular degeneration (AMD), the method comprising the steps of (a) selecting a subject that has one or more CFH, C3, and/or CFB gene mutations; and (b) administering to the subject a complement factor H (CFH) polypeptide or biologically active fragment and/or variant thereof.

In another aspect, the instant disclosure provides a method of treating a subject having an ocular disease associated with neovascularization, the method comprising the steps of: (a) administering to the subject a complement factor H (CFH) polypeptide or biologically active fragment and/or variant thereof; and (b) administering to the subject a vascular endothelial growth factor (VEGF) antagonist. In certain embodiments, the VEGF is VEGF-A. It is understood that patients having ocular diseases associated with neovascularization, particularly those receiving a treatment of a VEGF antagonist, have a greater risk of developing geographic atrophy. Given the ability of CFH to inhibit the alternative pathway of complement activation, which plays an important role in geographic atrophy, it is contemplated that a combination therapy of CFH and a VEGF antagonist can be used to treat these patients. In certain embodiments, the ocular disease associated with neovascularization is age-related macular degeneration (AMD), for example, neovascular AMD. In certain embodiments, the ocular disease associated with neovascularization is diabetic retinopathy, for example, diabetic macular edema. In certain embodiments, the subject has one or more CFH, C3, and/or CFB gene mutations.

A wide variety of diseases of the eye associated with one or more CFH mutations may be treated or prevented using the methods provided herein. Diseases of the eye that may be treated or prevented using the methods of the disclosure include but are not limited to, glaucoma, macular degeneration (e.g., age-related macular degeneration), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, systemic infections, or underlying carotid disease, a toxic compound or agent, or photically).

### General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N.Y. (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, NY (2003); Short Protocols in Molecular Biology (Wiley and Sons, 1999). The skilled worker would recognize many additional sources describing conventional techniques of molecular biology (including recombinant techniques), microbiology, cell biology, biochemistry and immunology.

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the disclosure.

Exemplary methods and materials are described herein, although methods and materials similar or equivalent to those described herein can also be used in the practice or testing of the present disclosure. The materials, methods, and examples are illustrative only and not intended to be limiting.

### Definitions

The following terms, unless otherwise indicated, shall be understood to have the following meanings:

As used herein, “residue” refers to a position in a protein and its associated amino acid identity.

As used herein, and unless otherwise stated, the term “CFH” or “Complement Factor H” encompasses complement factor H and factor-H-like protein 1 (FHL1). Exemplary sequences of CFH are SEQ ID NOs: 1-4, with SEQ ID NO: 4 corresponding to the FHL1 amino acid sequence.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR', CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide,” “oligopeptide,” “peptide,” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, a “host cell” includes an individual cell or cell culture that can be or has been a recipient for vector(s) for incorporation of polynucleotide inserts. In the alternative, the term “host cell” may refer to the target cell in which expression of the transgene is desired.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The terms “patient,” “subject,” or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, 45, 50, 55, 60, 65, 70, 75, 80, 85, 90 or 95 years of age.

In one embodiment, the subject has, or is at risk of developing a disease of the eye. A disease of the eye, includes, without limitation, AMD, retinitis pigmentosa, rod-cone dystrophy, Leber's congenital amaurosis, Usher's syndrome, Bardet-Biedl Syndrome, Best disease, retinoschisis, Stargardt disease (autosomal dominant or autosomal recessive), untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In another embodiment, the subject has, or is at risk of developing glaucoma, Leber's hereditary optic neuropathy, lysosomal storage disorder, or peroxisomal disorder. In another embodiment, the subject has shown clinical signs of a disease of the eye.

In some embodiments, the subject has, or is at risk of developing a renal disease or complication. In some embodiments, the renal disease or complication is associated with AMD or aHUS.

In some embodiments, the subject has, or is at risk of developing AMD or aHUS.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one embodiment, the subject shows degeneration of the outer nuclear layer (ONL). In another embodiment, the subject has been diagnosed with a disease of the eye. In yet another embodiment, the subject has not yet shown clinical signs of a disease of the eye.

As used herein, the terms “prevent,” “preventing,” and “prevention” refer to the prevention of the recurrence or onset of, or a reduction in one or more symptoms of a disease or condition (e.g., a disease of the eye) in a subject as result of the administration of a therapy (e.g., a prophylactic or therapeutic agent). For example, in the context of the administration of a therapy to a subject for an infection, “prevent,” “preventing,” and “prevention” refer to the inhibition or a reduction in the development or onset of a disease or condition (e.g., a disease of the eye), or the prevention of the recurrence, onset, or development of one or more symptoms of a disease or condition (e.g., a disease of the eye), in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition (e.g., a disease of the eye), treatment refers to the reduction or amelioration of the progression, severity, and/or duration of an infection (e.g., a disease of the eye or symptoms associated therewith), or the amelioration of one or more symptoms resulting from the administration of one or more therapies (including, but not limited to, the administration of one or more prophylactic or therapeutic agents).

“Administering” or “administration of” a substance, a compound or an agent to a subject can be carried out using one of a variety of methods known to those skilled in the art. For example, a compound or an agent can be administered intravitreally or subretinally. In particular embodiments, the compound or agent is administered intravitreally. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, the term “ocular cells” refers to any cell in, or associated with the function of, the eye. The term may refer to any one or more of photoreceptor cells, including rod, cone and photosensitive ganglion cells, retinal pigment epithelium (RPE) cells, glial cells, Muller cells, bipolar cells, horizontal cells, amacrine cells. In one embodiment, the ocular cells are bipolar cells. In another embodiment, the ocular cells are horizontal cells. In another embodiment, the ocular cells are ganglion cells. In particular embodiments, the cells are RPE cells.

Each embodiment described herein may be used individually or in combination with any other embodiment described herein.

### Complement Factor H

The disclosure provides a Complement Factor H (CFH) polypeptide or a biologically active fragment and/or variant thereof for use in any of the methods disclosed herein.

Bruch's membrane is a sheet of extracellular matrix that separates the retina from the underlying choroid, a highly vascularized layer that supplies oxygen and nutrition to the outer retina. Proteins up to 100 kDa can pass across Bruch's membrane, and proteins larger than 100 kDa can pass across to a variable extent. Additionally, the permeability of Bruch's membrane decreases with aging. In some embodiments, any of the CFH polypeptides or biologically active fragment and/or variant thereof disclosed herein is capable of diffusing across the Bruch's membrane. This may be accomplished by varying one or more of the following parameters: hydrodynamic size, dynamic radius, shape, post-translational modifications (e.g. glycosylation), net charge or propensity for the polypeptide to interact with Bruch's membrane components.

To prevent host cell and tissue destruction, the alternative pathway must be tightly controlled using a group of alternative pathway regulators. One endogenous mechanism often employed to regulate excessive alternative pathway activity is to prevent convertase formation completely by degrading the core component C3b to a proteolytic by-product that is incapable of forming the convertase. The C3b-degrading activity is primarily catalyzed by the protease complement factor I (CFI). However, CFI needs to form a binary complex with a second protein such as CFH in order to display its catalytic function. In some embodiments, any of the CFH polypeptides or biologically active fragments and/or variants thereof disclosed herein is capable of binding C3b. In some embodiments, the binding of CFH to C3b prevents the formation of a membrane attack complex. In some embodiments, the CFH polypeptide or biologically active fragment and/or variant thereof is capable of facilitating the breakdown of C3b. In some embodiments, the CFH polypeptide or biologically active fragment and/or variant thereof is capable of destabilizing C3bBb. In some embodiments, the CFH polypeptide or biologically active fragment and/or variant thereof competes with factor B for binding to C3b. In some embodiments, the CFH polypeptide or biologically active fragment and/or variant thereof prevents the formation of a C3 convertase (e.g. C3bBb). In some embodiments, the CFH polypeptide or biologically active fragment and/or variant thereof accelerates the decay of convertase complexes (e.g. C3bBb). In some embodiments, the CFH polypeptide or biologically active fragment and/or variant thereof accelerates the decay of the alternative pathway C5 convertase (C3b2Bb). In some embodiments, the CFH polypeptide or biologically active fragment and/or variant thereof is capable of suppressing C3b amplification. In some embodiments, the CFH polypeptide or biologically active fragment and/or variant thereof is capable of binding to a cell surface (e.g., an erythrocyte and/or endothelial cell). In some embodiments, the CFH polypeptide or biologically active fragment and/or variant thereof is capable of binding to heparin.

Human mature wildtype CFH is a 1213 amino acid soluble protein which comprises 20 complement-control protein modules (CCPs 1-20), which are approximately 60 amino acid residues in length. Alignment of the 20 CCPs demonstrates four invariant cysteine residues arranged in two conserved disulfide bonds, and a near-invariant tryptophan residue. Short three to eight amino acid residue “linkers” are found between the last residue of one CCP and the first residue of the next CCP. Each of the CCPs fold into a distinct three-dimensional (3-sheet rich structure (Schmidt C. Q. Clin Exp Immunol. 2008;151(1):14-24).

In some embodiments, the CFH polypeptide comprises at least one CCP module or a biologically active fragment and/or variant thereof. In some embodiments, the CFH polypeptide comprises at least two CCP modules or a biologically active fragment and/or variant thereof. In some embodiments, the CFH polypeptide comprises at least three CCP modules or a biologically active fragment and/or variant thereof. In some embodiments, the CFH polypeptide comprises at least four CCP modules or a biologically active fragment and/or variant thereof. In some embodiments, the CFH polypeptide comprises at least five CCP modules or a biologically active fragment and/or variant thereof. In some embodiments, the CFH polypeptide comprises at least six CCP modules or a biologically active fragment and/or variant thereof. In some embodiments, the CFH polypeptide comprises at least seven CCP modules or a biologically active fragment and/or variant thereof. In some embodiments, the CFH polypeptide comprises at least eight CCP modules or a biologically active fragment and/or variant thereof. In some embodiments, the CFH polypeptide comprises at least nine CCP modules or a biologically active fragment and/or variant thereof. In some embodiments, the CFH polypeptide comprises at least ten CCP modules or a biologically active fragment and/or variant thereof. In some embodiments, the CFH polypeptide comprises at least eleven CCP modules or a biologically active fragment and/or variant thereof. In some embodiments, the CFH polypeptide comprises at least twelve CCP modules or a biologically active fragment and/or variant thereof. In some embodiments, the CFH polypeptide comprises at least thirteen CCP modules or a biologically active fragment and/or variant thereof. In some embodiments, the CFH polypeptide comprises at least fourteen CCP modules or a biologically active fragment and/or variant thereof. In some embodiments, the CFH polypeptide comprises at least fifteen CCP modules or a biologically active fragment and/or variant thereof. In some embodiments, the CFH polypeptide comprises at least sixteen CCP modules or a biologically active fragment and/or variant thereof. In some embodiments, the CFH polypeptide comprises at least seventeen CCP modules or a biologically active fragment and/or variant thereof. In some embodiments, the CFH polypeptide comprises at least eighteen CCP modules or a biologically active fragment and/or variant thereof. In some embodiments, the CFH polypeptide comprises at least nineteen CCP modules or a biologically active fragment and/or variant thereof. In some embodiments, the CFH polypeptide comprises twenty CCP modules or a biologically active fragment and/or variant thereof.

In some embodiments, the CFH polypeptide comprises any one of or any combination of CCP modules 1, 2, 3, 4, 5, 6, 7, 8, 9, 10, 11, 12, 13, 14, 15, 16, 17, 18, 19, and/or 20 or a biologically active fragment and/or variant thereof. In some embodiments, the CFH polypeptide comprises at least one of CCP modules 1, 2, 3, 4, 5, 6, 7, 8, 9, 10, 11, 12, 13, 14, 15, 16, 17, 18, 19, and/or 20 or a biologically active fragment and/or variant thereof. In some embodiments, the CFH polypeptide comprises CCP modules 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, or 1-20 or a biologically active fragment and/or variant thereof. In some embodiments, the CFH polypeptide comprises CCP modules 2-20, 3-20, 4-20, 5-20, 6-20, 7-20, 8-20, 9-20, 10-20, 11-20, 12-20, 13-20, 14-20, 15-20, 16-20, 17-20, 18-20, or 19-20 or a biologically active fragment and/or variant thereof. In some embodiments, the CFH polypeptide comprises CCP modules 1-2 and 19-20, or a biologically active fragment and/or variant thereof. In some embodiments, the CFH polypeptide comprises CCP modules 1-4 and 19-20, or a biologically active fragment and/or variant thereof. In some embodiments, the CFH polypeptide comprises CCP modules 1-2 and 18-20, or a biologically active fragment and/or variant thereof. In some embodiments, the CFH polypeptide comprises CCP modules 1-4 and 18-20, or a biologically active fragment and/or variant thereof.

In some embodiments, the CFH polypeptide comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 1, or a biologically active fragment thereof. In some embodiments, the CFH polypeptide comprises an amino acid sequence that is at least 90% identical to the amino acid sequence of SEQ ID NO: 1, or a biologically active fragment thereof. In some embodiments, the CFH polypeptide comprises an amino acid sequence that is at least 95% identical to the amino acid sequence of SEQ ID NO: 1, or a biologically active fragment thereof.

In some embodiments, the CFH polypeptide comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 2, or a biologically active fragment thereof. In some embodiments, the CFH polypeptide comprises an amino acid sequence that is at least 90% identical to the amino acid sequence of SEQ ID NO: 2, or a biologically active fragment thereof. In some embodiments, the CFH polypeptide comprises an amino acid sequence that is at least 95% identical to the amino acid sequence of SEQ ID NO: 2, or a biologically active fragment thereof.

In some embodiments, the CFH polypeptide comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 3, or a biologically active fragment thereof. In some embodiments, the CFH polypeptide comprises an amino acid sequence that is at least 90% identical to the amino acid sequence of SEQ ID NO: 3, or a biologically active fragment thereof. In some embodiments, the CFH polypeptide comprises an amino acid sequence that is at least 95% identical to the amino acid sequence of SEQ ID NO: 3, or a biologically active fragment thereof.

In some embodiments, the CFH polypeptide is an FHL1 polypeptide that comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 4, or a biologically active fragment thereof. In some embodiments, the CFH polypeptide comprises an amino acid sequence that is at least 90% identical to the amino acid sequence of

SEQ ID NO: 4, or a biologically active fragment thereof. In some embodiments, the CFH polypeptide comprises an amino acid sequence that is at least 95% identical to the amino acid sequence of SEQ ID NO: 4, or a biologically active fragment thereof.

In some embodiments, the CFH polypeptide comprises at least CCP modules 1-4 or a biologically active fragment and/or variant thereof. In some embodiments, the CFH polypeptide comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 5, or a biologically active fragment thereof. In some embodiments, the CFH polypeptide comprises an amino acid sequence that is at least 90% identical to the amino acid sequence of SEQ ID NO: 5, or a biologically active fragment thereof. In some embodiments, the CFH polypeptide comprises an amino acid sequence that is at least 95% identical to the amino acid sequence of SEQ ID NO: 5, or a biologically active fragment thereof.

In some embodiments, the CFH polypeptide comprises at least CCP modules 1-5 or a biologically active fragment and/or variant thereof. In some embodiments, the CFH polypeptide comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 6, or a biologically active fragment thereof. In some embodiments, the CFH polypeptide comprises an amino acid sequence that is at least 90% identical to the amino acid sequence of SEQ ID NO: 6, or a biologically active fragment thereof. In some embodiments, the CFH polypeptide comprises an amino acid sequence that is at least 95% identical to the amino acid sequence of SEQ ID NO: 6, or a biologically active fragment thereof.

In some embodiments, the CFH polypeptide comprises at least CCP modules 1-7 or a biologically active fragment and/or variant thereof. In some embodiments, the CFH polypeptide comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 7, or a biologically active fragment thereof. In some embodiments, the CFH polypeptide comprises an amino acid sequence that is at least 90% identical to the amino acid sequence of SEQ ID NO: 7, or a biologically active fragment thereof. In some embodiments, the CFH polypeptide comprises an amino acid sequence that is at least 95% identical to the amino acid sequence of SEQ ID NO: 7, or a biologically active fragment thereof.

In some embodiments, the CFH polypeptide comprises at least CCP modules 19-20 or a biologically active fragment and/or variant thereof. In some embodiments, the CFH polypeptide comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 8, or a biologically active fragment thereof. In some embodiments, the CFH polypeptide comprises an amino acid sequence that is at least 90% identical to the amino acid sequence of SEQ ID NO: 8, or a biologically active fragment thereof. In some embodiments, the CFH polypeptide comprises an amino acid sequence that is at least 95% identical to the amino acid sequence of SEQ ID NO: 8, or a biologically active fragment thereof.

In some embodiments, the CFH polypeptide comprises at least CCP modules 18-20 or a biologically active fragment and/or variant thereof. In some embodiments, the CFH polypeptide comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 9, or a biologically active fragment thereof. In some embodiments, the CFH polypeptide comprises an amino acid sequence that is at least 90% identical to the amino acid sequence of SEQ ID NO: 9, or a biologically active fragment thereof. In some embodiments, the CFH polypeptide comprises an amino acid sequence that is at least 95% identical to the amino acid sequence of SEQ ID NO: 9, or a biologically active fragment thereof.

In some embodiments, the CFH polypeptide or biologically active fragment and/or variant thereof is encoded by a nucleotide sequence that is at least 70%, 75%, 80%, 85%, 90%, 91%, 92%, 93%, 94%, 95%, 96%, 97%, 98%, 99% or 100% identical to the nucleotide sequence of any one of SEQ ID NOs: 10-13.

In some embodiments, the biologically active fragment and/or variant is capable of inducing any one or more of the effects listed above with regard to a wildtype CFH protein. For example, in some embodiments, the biologically active fragment and/or variant of CFH is capable of acting as a cofactor with CFI to facilitate C3b cleavage. In some embodiments, the CFH protein or biologically active fragment and/or variant thereof is capable of diffusing across the Bruch's membrane. In some embodiments, the CFH protein or biologically active fragment and/or variant thereof is capable of binding C3b. In some embodiments, the CFH protein or biologically active fragment and/or variant thereof is capable of facilitating the breakdown of C3b. In some embodiments, the CFH protein or biologically active fragment and/or variant thereof is capable of binding to a cell surface (e.g., an erythrocyte and/or endothelial cell). In some embodiments, the CFH protein or biologically active fragment and/or variant thereof is capable of binding to heparin. In some embodiments, the CFH protein or biologically active fragment and/or variant thereof is capable of reducing C5b9 levels generated as a result of complement activation (e.g., as measured in a Wieslab AP assay (see, e.g., Example 1). In some embodiments, the CFH protein or biologically active and/or variant fragment thereof is capable of inhibiting hemolysis. In some embodiments, the biologically active fragment and/or variant is at least 200, 250, 300, 350, 400, 450, 500, 600, 700, 800, 900, 1000, 1100, 1200, or 1213 amino acids in length. In some embodiments, the biologically active fragment and/or variant is between 100-1213, 200-1213, 300-1213, 400-1213, 500-1213, 600-1213, 700-1213, 800-1213, 900-1213, 1000-1213, 1100-1213, 500-1213, 100-200, 100-300, 100-400, 100-500, 100-600, 100-700, 100-800, 100-900, 200-300, 200-400, 200-500, 200-600, 200-700, 200-800, 200-900, 300-400, 300-500, 300-600, 300-700, 300-800, 300-900, 400-500, 400-600, 400-700, 400-800, 400-900, 500-600, 500-700, 500-800, 500-900, 600-700, 600-800, 600-900, 700-800, 700-900, or 800-900 amino acids in length. In some embodiments, the biologically active fragment comprises at least 100, 150, 200, 250, 300, 350, 400, 450, 500, 550, 600, 650, 700, 750, 800, 850, 900, 950, 1000, 1050, 1100, 1150, or 1200 consecutive amino acids from a sequence that is at least 70%, 75%, 80%, 85%, 90%, 91%, 92%, 93%, 94%, 95%, 96%, 97%, 98%, 99% or 100% identical to the amino acid sequence of SEQ ID NO: 1. In some embodiments, the biologically active fragment and/or variant comprises at least 100, 150, 200, 250, 300, 350, 400, 450, 500, 550, 600, 650, 700, 750, 800, 850, 900, 950, 1000, 1050, 1100, 1150, or 1200 consecutive amino acids of SEQ ID NO: 1.

In some embodiments, any of the CFH polypeptides or biologically active fragments and/or variants disclosed herein is a modified CFH polypeptide or biologically active fragment thereof as compared to a reference CFH sequence (e.g., a protein comprising the amino acid sequence of any one of SEQ ID NOs: 1-4). In some embodiments, a modified CFH polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains (e.g., one or more CCP domains) or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features (e.g., insertion of a linker). A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gln (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), Ile (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He; (2) polar without charge: Cys, Ser, Thr, Asn, Gin; (3) acidic (negatively charged): Asp, Glu; (4) basic (positively charged): Lys, Arg; and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile; (2) Polar without charge: Cys, Ser, Thr, Asn, Gln; (3) Acidic (negatively charged): Asp, Glu; (4) Basic (positively charged): Lys, Arg; (5) Residues that influence chain orientation: Gly, Pro; and (6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine, N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, y-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and amino acids (e.g., 4-hydroxyproline). In one aspect, a modified CFH protein or biologically active fragment thereof comprises 1, 2, 3, 4, 5, 6, 7, 8, 9, 10, 11, 12, 13, 14, or 15 conservative or non-conservative substitutions relative to the wild-type CFH polypeptide or biologically active fragment thereof (e.g., a polypeptide having the amino acid sequence of any one of SEQ ID NOs: 1-4).

In some embodiments, the CFH polypeptide or biologically active fragment and/or variant thereof is a precursor CFH polypeptide or biologically active fragment and/or variant thereof. In some embodiments, the precursor CFH polypeptide or biologically active fragment and/or variant thereof is processed to a mature CFH polypeptide or biologically active fragment and/or variant thereof after administration to a subject. In some embodiments, the CFH polypeptide or biologically active fragment and/or variant thereof is a mature CFH polypeptide or biologically active fragment and/or variant thereof.

In certain embodiments, any of the CFH polypeptides or biologically active fragments and/or variants disclosed herein may further comprise post-translational modifications in addition to any that are naturally present in the native polypeptides. Such modifications include, but are not limited to, acetylation, carboxylation, glycosylation, phosphorylation, lipidation, pegylation (polyethylene glycol) and acylation. As a result, the modified polypeptides may contain non-amino acid elements, such as polyethylene glycols, lipids, mono- or poly-saccharides, and phosphates. Effects of such non-amino acid elements on the functionality of a polypeptide may be tested as described herein for other polypeptide variants. When a polypeptide is produced in cells by cleaving a nascent form of the polypeptide, post-translational processing may also be important for correct folding and/or function of the protein. Different cells have specific cellular machinery and characteristic mechanisms for such post-translational activities and may be chosen to ensure the correct modification and processing of the polypeptides.

### Pharmaceutical Compositions and Routes of Administration

Also provided herein are pharmaceutical compositions comprising any of the CFH polypeptides or biologically active fragments and/or variants thereof disclosed herein, and a pharmaceutically acceptable carrier. The pharmaceutical compositions may be suitable for any mode of administration described herein; for example, by intravitreal administration.

In some embodiments, the composition comprises a CFH polypeptide or biologically active fragment and/or variant thereof.

In some embodiments, the pharmaceutical compositions comprising a CFH polypeptide or biologically active fragment and/or variant thereof described herein and a pharmaceutically acceptable carrier is suitable for administration to a human subject. Such carriers are well known in the art (see, e.g., Remington's Pharmaceutical Sciences, 15th Edition, pp. 1035-1038 and 1570-1580). In some embodiments, the pharmaceutical compositions comprising a CFH polypeptide or biologically active fragment and/or variant thereof described herein and a pharmaceutically acceptable carrier is suitable for ocular injection. In some embodiments, the pharmaceutical composition is suitable for intravitreal injection. In some embodiments, the pharmaceutical composition is suitable for subretinal delivery. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, the CFH polypeptide or biologically active fragment and/or variant thereof is formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal or intravitreal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the CFH polypeptide or biologically active fragment and/or variant thereof is to be stored long-term, it may be frozen in the presence of glycerol or Tween20. In another embodiment, the pharmaceutically acceptable carrier comprises a surfactant, such as perfluorooctane (Perfluoron liquid).

In certain embodiments of the methods described herein, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parenteral routes of administration. Routes of administration may be combined, if desired. In certain embodiments, the pharmaceutical compositions of the disclosure are administered after administration of an initial loading dose of the CFH polypeptide or biologically active fragment and/or variant thereof.

In some embodiments, any of the CFH polypeptides or biologically active fragments and/or variants thereof or pharmaceutical compositions disclosed herein are administered to a patient such that they target cells of any one or more layers or regions of the retina or macula. For example, the compositions disclosed herein target cells of any one or more layers of the retina, including the inner limiting membrane, the nerve fiber layer, the ganglion cell layer (GCL), the inner plexiform layer, the inner nuclear layer, the outer plexiform layer, the outer nuclear layer, the external limiting membrane, the layer of rods and cones, or the retinal pigment epithelium (RPE). In some embodiments, the compositions disclosed herein target glial cells of the GCL, Muller cells, and/or retinal pigment epithelial cells. In some embodiments, the compositions disclosed herein targets cells of any one or more regions of the macula including, for example, the umbo, the foveolar, the foveal avascular zone, the fovea, the parafovea, or the perifovea. In some embodiments, the route of administration does not specifically target neurons. In some embodiments, the route of administration is chosen such that it reduces the risk of retinal detachment in the patient (e.g., intravitreal rather than subretinal administration). In some embodiments, intravitreal administration is chosen if any of the CFH polypeptides or biologically active fragments and/or variants thereof disclosed herein is to be administered to an elderly adult (e.g., at least 60 years of age). In particular embodiments, any of the CFH polypeptides or biologically active fragments and/or variants thereof or pharmaceutical compositions disclosed herein are administered to a subject intravitreally. Procedures for intravitreal injection are known in the art (see, e.g., Peyman, G. A., et al. (2009) Retina 29(7):875-912 and Fagan, X.J. and Al-Qureshi, S. (2013) Clin. Experiment. Ophthalmol. 41(5):500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues (e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limbus in pseudophakic or aphakic patients, and 3.5-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antiobiotic. The eye may also be rinsed to remove excess sterilizing agent.

Furthermore, in certain embodiments it is desirable to perform non-invasive retinal imaging and functional studies to identify areas of specific ocular cells to be targeted for therapy. In these embodiments, clinical diagnostic tests are employed to determine the precise location(s) for one or more subretinal injection(s). These tests may include ophthalmoscopy, electroretinography (ERG) (particularly the b-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc.

These, and other desirable tests, are described in International Patent Application No. PCT/US2013/022628. In view of the imaging and functional studies, in some embodiments, one or more injections are performed in the same eye in order to target different areas of retained bipolar cells. The volume and concentration of the CFH polypeptide or biologically active fragment and/or variant thereof for each injection is determined individually, as further described below, and may be the same or different from other injections performed in the same, or contralateral, eye. In another embodiment, a single, larger volume injection is made in order to treat the entire eye. In one embodiment, the volume and concentration of the CFH polypeptide or biologically active fragment and/or variant thereof composition is selected so that only a specific region of ocular cells is impacted. In another embodiment, the volume and/or concentration of the CFH polypeptide or biologically active fragment and/or variant thereof composition is a greater amount, in order reach larger portions of the eye, including non-damaged ocular cells.

The composition may be delivered in a volume of from about 0.1 μL to about 1 mL, including all numbers within the range, depending on the size of the area to be treated, the route of administration, and the desired effect of the method. In some embodiments, the volume is between 25-100 μL . In some embodiments, the volume is between 40-60 μL. In one embodiment, the volume is about 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. It is desirable that the lowest effective concentration of CFH polypeptide or biologically active fragment and/or variant thereof be utilized in order to reduce the risk of undesirable effects, such as toxicity, retinal dysplasia and detachment. Still other dosages and administration volumes in these ranges may be selected by the attending physician, taking into account the physical state of the subject, preferably human, being treated, the age of the subject, the particular ocular disorder and the degree to which the disorder, if progressive, has developed. For extra-ocular delivery, the dosage will be increased according to the scale-up from the retina.

### Methods of Treatment/Prophylaxis

In some embodiments, the disclosure provides a method for treating a subject having a disease or disorder, wherein the subject has one or more CFH mutations. A subject “has” a CFH mutation if DNA from a sample (e.g., a blood sample or a sample from the patient's eye) from the subject is determined to carry one or more CFH mutations. In some embodiments, any of the methods disclosed herein are for treating a subject in whom it has been determined has one or more CFH mutations. In some embodiments, the presence or absence of any of the CFH mutations disclosed herein is determined by genetic testing.

Described herein are various methods of preventing, treating, arresting progression of or ameliorating the ocular disorders and retinal changes associated therewith. Generally, the methods include administering to a mammalian subject in need thereof, an effective amount of a composition comprising a CFH polypeptide or biologically active fragment and/or variant thereof as described above, and a pharmaceutically acceptable carrier. Any of the CFH polypeptides or biologically active fragments and/or variants thereof described herein are useful in the methods described below.

In some embodiments, CFH polypeptide or biologically active fragment and/or variant thereof are administered locally to the cells in the retina for treating diseases such as leber congenital amaurosis (LCA), retinitis pigmentosa, and age-related macular degeneration. The cells that will be the treatment target in these diseases are either the photoreceptor cells in the retina or the cells of the RPE underlying the neurosensory retina. Delivering CFH polypeptide or biologically active fragment and/or variant thereof to these cells may be by injection into the subretinal space between the retina and the RPE. In some embodiments, the disclosure provides methods to deliver CFH polypeptide or biologically active fragment and/or variant thereof to cells of the retina.

In a certain aspect, the disclosure provides a method of treating a subject having age-related macular degeneration (AMD), comprising the step of administering to the subject any of the CFH polypeptides or biologically active fragments and/or variants thereof of the disclosure. In certain embodiments, the pharmaceutical compositions of the disclosure comprise a pharmaceutically acceptable carrier. In certain embodiments, the pharmaceutical compositions of the disclosure comprise PBS. In certain embodiments, the pharmaceutical compositions of the disclosure comprise pluronic. In certain embodiments, the pharmaceutical compositions of the disclosure comprise PBS, NaCl and pluronic. In certain embodiments, the CFH polypeptides or biologically active fragments and/or variants thereof are administered by intravitreal injection in a solution of PBS with additional NaCl and pluronic.

In some embodiments, any of the CFH polypeptides or biologically active fragments and/or variants thereof disclosed herein is administered to cell(s) or tissue(s) in a test subject. In some embodiments, the cell(s) or tissue(s) in the test subject express less CFH, or less functional CFH, than expressed in the same cell type or tissue type in a reference control subject or population of reference control subjects. In some embodiments, the reference control subject or population of reference control subjects does not have any of the CFH mutations disclosed herein. In some embodiments, the reference control subject or population of reference control subjects does not have a mutation that impairs CFH function. In some embodiments, the reference control subject is of the same age and/or sex as the test subject. In some embodiments, the reference control subject is a healthy subject, e.g., the subject does not have a disease or disorder of the eye. In some embodiments, the reference control subject does not have a disease or disorder of the eye associated with activation of the complement cascade. In some embodiments, the reference control subject does not have macular degeneration. In some embodiments, the eye or a specific cell type of the eye (e.g., cells in the foveal region) in the test subject expresses at least 95%, 90%, 80%, 70%, 60%, 50%, 40%, 30%, 20%, 10%, 5%, or 1% less CFH or functional CFH as compared to the levels in the reference control subject or population of reference control subjects. In some embodiments, the eye or a specific cell type of the eye (e.g., cells in the foveal region) in the test subject express CFH protein having any one or more of the CFH mutations disclosed herein. In some embodiments, the eye or a specific cell type of the eye (e.g., cells in the foveal region) in the reference control subject do not express a CFH protein having any of the CFH mutations disclosed herein. In some embodiments, administration of any of the CFH polypeptides or biologically active fragments and/or variants thereof disclosed herein to the cell(s) or tissue(s) of the test subject results in an increase in levels of functional CFH protein. In some embodiments, administration of any of the CFH polypeptides or biologically active fragments and/or variants thereof disclosed herein to the cell(s) or tissue(s) of the test subject results in an increase in levels of functional CFH protein such that the increased levels are within 90%, 80%, 70%, 60%, 50%, 40%, 30%, 20%, 10%, 5%, or 1% of, or are the same as, the levels functional CFH protein expressed by the same cell type or tissue type in the reference control subject or population of reference control subjects. In some embodiments, administration of any of the CFH polypeptides or biologically active fragments and/or variants thereof disclosed herein to the cell(s) or tissue(s) of the test subject results in an increase in levels of functional CFH protein, but the increased levels of functional CFH protein do not exceed the levels of functional CFH protein expressed by the same cell type or tissue type in the reference control subject or population of reference control subjects. In some embodiments, administration of any of the CFH polypeptides or biologically active fragments and/or variants thereof disclosed herein to the cell(s) or tissue(s) of the test subject results in an increase in levels functional CFH protein, but the increased levels of functional CFH protein exceed the levels functional CFH protein by no more than 1%, 5%, 10%, 20%, 25%, 30%, 40%, 50%, 60%, 70%, 80%, 90% or 100% of the levels expressed by the same cell type or tissue type in the reference control subject or population of reference control subjects.

In some embodiments, any of the treatment and/or prophylactic methods disclosed herein are applied to a subject. In some embodiments, the subject is a mammal. In some embodiments, the subject is a human. In some embodiments, the human is an adult. In some embodiments, the human is an elderly adult. In some embodiments, the human is at least 40, 45, 50, 55, 60, 65, 70, 75, 80, 85, 90, or 95 years of age. In particular embodiments, the human is at least 60 or 65 years of age.

In some embodiments, any of the treatment and/or prophylactic methods disclosed herein is for use in treatment of a patient having one or more CFH mutations. In some embodiments, any of the treatment and/or prophylactic methods disclosed herein is for use in treatment of a patient having one or more CFH mutations that causes macular degeneration (AMD) or that increases the likelihood that a patient develops AMD. In some embodiments, any of the treatment and/or prophylactic methods disclosed herein is for use in treatment of a patient having one or more mutations that causes atypical hemolytic uremic syndrome (aHUS) or that increases the likelihood that a patient develops aHUS. In some embodiments, the one or more mutations are in the patient's CFH gene. In some embodiments, the subject has a loss-of-function mutation in the subject's CFH gene.

In some embodiments, any of the treatment and/or prophylactic methods disclosed herein is for use in treatment of a patient having one or more mutations in the patient's CFH gene. In some embodiments, the treatment and/or prophylactic method is for use in treating a patient in whom it has been determined has one or more of any of the CFH mutations disclosed herein. In some embodiments, the patient has a mutation in one or more of CCP domains 1-20, or any combination thereof. In some embodiments, the patient has a mutation in one or more of CCP domains 1-2 or 18-20. In some embodiments, the patient has a mutation in CCP1. In some embodiments, the patient has a mutation in CCP2. In some embodiments, the patient has a mutation in CCP3. In some embodiments, the patient has a mutation in CCP4. In some embodiments, the patient has a mutation in CCPS. In some embodiments, the patient has a mutation in CCP6. In some embodiments, the patient has a mutation in CCP7. In some embodiments, the patient has a mutation in CCPR. In some embodiments, the patient has a mutation in CCP9. In some embodiments, the patient has a mutation in CCP10. In some embodiments, the patient has a mutation in CCP11. In some embodiments, the patient has a mutation in CCP12. In some embodiments, the patient has a mutation in CCP13. In some embodiments, the patient has a mutation in CCP14. In some embodiments, the patient has a mutation in CCP15. In some embodiments, the patient has a mutation in CCP16. In some embodiments, the patient has a mutation in CCP17. In some embodiments, the patient has a mutation in CCP18. In some embodiments, the patient has a mutation in CCP19. In some embodiments, the patient has a mutation in CCP20. In some embodiments, the patient has one or more mutations in the disulphide bond sites in the CFH protein. In some embodiments, the mutation is one or more of the mutations selected from the group consisting of: H402Y, G69E, D194N, W314C, A806T, Q950H, p.Ile184fsX, p.Lys204fsX, c.1697-17_-8de1, A161S, A173G, R175Q, V62I, V1007L, S890I, S193L, 1216T, A301Nfs*25 (i.e., amino acid A at position 301 changed to amino acid N by a frameshifting mutation, which also leads to translation termination 25 residues downstream), W379R, Q400K, Q950H, T956M, R1210C, N1050Y, E936D, Q408X, R1078S, c.350+6T->G, R567G, R53C, R53H, R2T, A892V, R567G, I221V, S159N, P562H, F960S, R303W, R303Q, K666N, G1194D, P258L, G650V, D130N, S58A, R166W, R232Q, R127H, K1202N, G397Stop, Stop450R, R830W, I622L, T732M, S884Y, L24V, Y235H, K527N, R582H, C973Y, V1089M, E123G, T291S, R567K, E625Stop, N802S, N1056K, R1203W, Q1076E, P26S, T46A, T91S, C129Y, R166Q, E167Q, R175P, C192F, W198*, V206M, G218*, M239T, Y277*, C325Y, R341H, R364L, P384R, C431S, D454A, A473V, P503A, N516K, I551T, H699R, F717L, W978R, P981S, A1010V, W1037*, P1051L, I1059T, Q1143E, R1206H, T1227I, L24V, H169R, R257H, K410E, V609I, D619N, A892V, G1002R, G278S, T30*, I32Stop, R78G, Q81P, V111E, W134R, P139S, M162V, E189Stop, K224De1, K224De1, A307A, H332Y, S411T, C448Y, L479Stop, R518T, T519A, C536R, C564P, C569Stop, L578Stop, P621T, C623S, C630W, E635D, K670T, Q672Q, C673Y, C673S, S714Stop, S722*, C733Y, V737V, E762Stop, N774Stop, R780I, G786*, M823T, V835L, E847V, E850K, C853R, C853T, C864S, C870R, H878H, I881L, E889Stop, H893R, Y899Stop, Y899D, C915S, C915Stop, W920R, Q925Stop, C926F, Y951H, C959Y, P968*, I970V, T987A, N997T, G1011*, T10171, Y1021F, C1043R, T1046T, V1054I, V1060A, V1060L, C1077W, T1097W, T1097T, D1119G, D1119N, P1130L, V1134G, E1135R, E1137L, E1139Stop, Y1142D, Y1142C, C1152S, W1157R, P1161T, C1163T, P1166L, V1168E, V1168Stop, I1169L, E1172Stop, Y1177C, R1182S, W1183L, W1183R, W1183L, W1183Stop, W1183C, T1184R, T1184A, K1186H, K1188De1, L1189R, L1189F, S1191L, S1191W, E1195Stop, V1197A, E1198A, E1198Stop, F1199S, V1200L, G1204E, L1207R, S1211P, R1215Q, R1215G, T1216De1, C1218R, Y1225*, P1226S, L3V, H821Y, E954de1, G255E, T1038R, V383A, V641A, P213A, I221V, E229K, R2T, R1072G, G967E, N819S, V579F, G19K, A18S, K834E, T504M, R662I, P668L, G133R, I184T, L697F, H1165Y, G1110A, pIle808_G1n809de1, 1760L, T447R, 1808M, I868M, L765F, N767S, R567G, K768N, S209L, Q628K, D214Y, N401D, I216K, Q464R, I777V, E229D, M823I, R232Ter, S266L, P260S, E23G, C80Y, R78T, R582H, N638D, N638S, P258L, L3F, R257H, G240R, G69R, D855N, M11I, K472N, Q840H, E850K, Y899H, T645M, M805V, K919T, E201G, V407A, I907L, T914K, H332R, V144M, S652G, D195N, C146S, P661R, E677Q, V482I, T34R, A421T, R281G, C509Y, K666N, P440S, C442G, N607D, A425V, G667E, P440L, I49V, R387G, E625K, E625Ter, T135S, P43S, K283E, I124V, T36V, I563T, G350E, D619G, T321I, T286A, P384L, T739N, M515L, V158A, G727R, T724K, F717L, M162V, C178R, G700R, A161T, F176L, R295S, F298Y, G297S, P300L, R1040K, V552L, T310I, T531A, G928D, Ter386RextTer 69â€, Q1143K, Y534C, P981L, K308N, D538E, R1215Ter, E105V, T10171, N1050I, P935S, Y951H, T1097M, D947H, E961D, G962S, G964E, 1970V, R1072T, P1114L, S1122T, F960C, R1074C, R1182T, R1074L, S884Y, S890T, V837I, V941F, V158I, D748V, I216T, H371N, L750F, P418T, M432V, D693N, A746E, V111E, c.2237-2A>G, P982S, V579A, E591D, V579I, V65I, P418S, Y1067C, D772N, V72L, E189K, A1027P, D798N, N61D, P384S, N521S, P1068S, E395K, N774S, H577R, E833K, K6E, H337R, R444C, L741F, Y42F, D288E, S705F, R1040G, D214H, N757D, I861M, G848E, P923S, E201K, E902A, R303Q, G366E, D538H, K82R, E721K, Y1008H, R1074P, A806S, Q807R, C389Y, H764Y, K867N, P392T, L394M, E456K, F459L, Y398C, E570K, D214N, I574V, I574T, G631C, T880I, V865F, V576A, N776S, P633S, N22D, P634A, N822I, R885S, R232L, E635D, R778K, L827V, C267R, Y779C, R582C, L77S, R257C, Y327H, N75K, L74F, S836T, Y243H, c.1519+5_1519+8delGT . . . , K507Q, A892S, 115T, P924L, A14V, N842K, G894R, G894E, Y271C, C9W, T504R, V683M, L385Pheâ€, S898R, Q408H, G409S, T34K, E648G, I412V, E338D, P799S, G480E, D798E, D195Y, R341C, D485H, D485G, K598Q, Y420H, P599T, N434H, R441T, C431G, V149A, V349I, T679A, P43T, G45D, R662G, T519I, L121P, P364L, P621A, H373Y, D538MfsTer14, H371P, T544A, T131A, R166G, V1771, V177A, R729S, F717V, N718S, S991G, L98I, Y1016Ter, T1217del, M1001T, K1004E, A1010T, G1011D, T1017A, T1031A, L1125F, R1203G, L1214M, W1096DfsTer20, H939N F960L, D966H, M1064I, E1071K, N1095K, T1106A, G1107E, C1109W, P1111S, V11971, Y1075F, S1079N, P1080S, E1082G, or Sto1232. In particular embodiments, the mutation is one or more of the mutations selected from the group consisting of: R2T, L3V, R53C, R53H, S58A, G69E, D90G, R175Q, S193L, I216T, I221V, R303W, H402Y, Q408X, P503A, G650V, R1078S, and R1210C. As used in the context of mutations, asterisk refers to a stop codon resulting in a C-terminal truncation. In some embodiments, any of the CFH mutant amino acid positions described herein correspond to the amino acid CFH sequence of SEQ ID NO: 2. In some embodiments, any of the CFH mutant amino acid positions described herein correspond to the amino acid CFH sequence of SEQ ID NO: 3.

In some embodiments, the patient has any one of the following CFH mutations: R2T, L3V, R53C, R53H, S58A, D90G, D130N, R175Q, R175P, I221V, R303W, R303Q, Q400K, Y402H, P503A, R567G, G650V, S890I, T956M, G1194D, or R1210C. In some embodiments, the patient has any one or more of the following CFH mutations: R2T, R53C, R53H, S58A, D130N, R175Q, R175P, I221V, R303W, R303Q, P503A, R567G, G650V, G1194D, or R1210C. In some embodiments, the patient has an R2T mutation. In some embodiments, the patient has an L3V mutation. In some embodiments, the patient has an R53C mutation. In some embodiments, the patient has an R53H mutation. In some embodiments, the patient has an S58A mutation. In some embodiments, the patient has a D90G mutation. In some embodiments, the patient has a D130N mutation. In some embodiments, the patient has an R175Q mutation. In some embodiments, the patient has an R175P mutation. In some embodiments, the patient has an I221V mutation. In some embodiments, the patient has an R303W mutation. In some embodiments, the patient has an R303Q mutation. In some embodiments, the patient has a Q400K mutation. In some embodiments, the patient has a Y402H mutation. In some embodiments, the patient has a P503A mutation. In some embodiments, the patient has an R567G mutation. In some embodiments, the patient has a G650V mutation. In some embodiments, the patient has an S890I mutation. In some embodiments, the patient has a T956M mutation. In some embodiments, the patient has a G1194D mutation. In some embodiments, the patient has an R1210C mutation. In some embodiments, any of the CFH mutant amino acid positions described herein correspond to the amino acid CFH sequence of SEQ ID NO: 2. In some embodiments, any of the CFH mutant amino acid positions described herein correspond to the amino acid CFH sequence of SEQ ID NO: 3.

In some embodiments, the patient is homozygous for any of the mutations disclosed herein. In some embodiments, the patient is heterozygous for any of the mutations disclosed herein. In particular embodiments, the patient expresses a mutant CFH protein, wherein the mutant CFH protein has reduced CFH activity as compared to a wildtype CFH protein (e.g., a CFH protein having the amino acid sequence of SEQ ID NO: 1, 2 or 3). In some embodiments, the CFH activity is the ability to bind to C3b. In some embodiments, the CFH activity is the ability to act as a cofactor with CFI and facilitate C3b cleavage. In some embodiments, the CFH activity is the ability to bind to a cell surface (e.g., an erythrocyte and/or endothelial cell). In some embodiments, the CFH activity is the ability to bind to heparin. In some embodiments, the CFH activity is the ability to inhibit C5b9 levels as a result of complement activation, e.g., as measured in a Wieslab AP assay (see, e.g., Example 1). In some embodiments, the CFH activity is the ability to inhibit hemolysis. In some embodiments, if the mutant CFH protein were tested in a functional assay, the mutant CFH protein would display reduced CFH activity as compared to a wildtype CFH protein (e.g., a CFH protein having the amino acid sequence of SEQ ID NO: 1, 2, or 3). Examples of CFH mutants associated with reduced CFH activity include R2T, R53C, R53H, S58A, D130N, R175Q, R175P, I221V, R303W, R303Q, P503A, R567G, G650V, G1194D, or R1210C CFH mutants. See, e.g., the functional assays in Example 1. In some embodiments, if a cell expresses less of a variant CFH polypeptide than the same cell would express a wildtype control CFH polypeptide, then the variant CFH is determined to be functionally impaired.

In some embodiments, if the subject has a CFH mutation that is associated with reduced CFH activity, then a greater amount of CFH polypeptide (or biologically active fragment and/or variant thereof) is administered to the subject than if the subject did not have a CFH mutation associated with reduced CFH activity in one or more activity assays. Examples of CFH mutants that do not have reduced activity in a cell expression assay, a C3b affinity assay, a decay acceleration assay, a cofactor assay, a hemolysis assay, or a Wieslab AP assay include L3V, D90G, Q400K, Y402H, S890I, or T956M CFH mutants. See, e.g., Example 1.

In some embodiments, the subject has one or more mutations in other complement pathway genes, optionally in combination with CFH mutations. It is understood that polymorphism is present in some complement pathway genes, resulting in multiple variants of the same gene. As used herein, the term “mutation” refers to a genetic variant or an amino acid sequence encoded by the genetic variant, even if the genetic variant is recognized as wildtype in certain populations. In some embodiments, the subject has one or more C3 mutations. In some embodiments, the subject has one or more C3 mutations selected from R102G, K155Q, V619M, and R735W. In some embodiments, the subject has one or more complement factor B (CFB) mutations. In some embodiments, the subject has the I242L mutation of CFB. In some embodiments, the subject has the 32R mutation of CFB. In some embodiments, the subject has the CFH 62V, C3 102G, and/or CFB 32R mutations. In some embodiments, the subject is homozygous for CFH 62V, C3 102G, and CFB 32R, which combination of homozygous mutations is predicted to have a prevalence of about 1.4% in the Caucasian population and is enriched in the AMD patient population.

In some embodiments, any of the methods disclosed herein are for treating a subject in whom it has been determined has one or more of any of the mutations disclosed herein.

In some embodiments, any of the CFH polypeptides or biologically active fragments and/or variants thereof disclosed herein are for use in treating a renal disease or complication. In some embodiments, the renal disease or complication is associated with AMD in the patient. In some embodiments, the renal disease or complication is associated with aHUS in the patient.

The retinal diseases described above are associated with various retinal changes. These may include a loss of photoreceptor structure or function; thinning or thickening of the outer nuclear layer (ONL); thinning or thickening of the outer plexiform layer (OPL); disorganization followed by loss of rod and cone outer segments; shortening of the rod and cone inner segments; retraction of bipolar cell dendrites; thinning or thickening of the inner retinal layers including inner nuclear layer, inner plexiform layer, ganglion cell layer and nerve fiber layer; opsin mislocalization; overexpression of neurofilaments; thinning of specific portions of the retina (such as the fovea or macula); loss of ERG function; loss of visual acuity and contrast sensitivity; loss of optokinetic reflexes; loss of the pupillary light reflex; and loss of visually guided behavior. In one embodiment, a method of preventing, arresting progression of or ameliorating any of the retinal changes associated with these retinal diseases is provided. As a result, the subject's vision is improved, or vision loss is arrested and/or ameliorated.

In a particular embodiment, a method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity.

In any of the methods described herein, the targeted cell may be an ocular cell. In one embodiment, the targeted cell is a glial cell. In one embodiment, the targeted cell is an RPE cell. In another embodiment, the targeted cell is a photoreceptor. In another embodiment, the photoreceptor is a cone cell. In another embodiment, the targeted cell is a Muller cell. In another embodiment, the targeted cell is a bipolar cell. In yet another embodiment, the targeted cell is a horizontal cell. In another embodiment, the targeted cell is an amacrine cell. In still another embodiment, the targeted cell is a ganglion cell. In still another embodiment, the gene may be expressed and delivered to an intracellular organelle, such as a mitochondrion or a lysosome.

As used herein “photoreceptor function loss” means a decrease in photoreceptor function as compared to a normal, non-diseased eye or the same eye at an earlier time point. In some embodiments, any of the methods disclosed herein may be used to increase photoreceptor function in a subject in need thereof. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using the functional studies described above and in the examples below, e.g., ERG or perimetry, which are conventional in the art.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured ocular cells, improve damage in injured ocular cells, or to provide enhanced vision. In one embodiment, the composition is administered before the disease becomes symptomatic or prior to photoreceptor loss. By symptomatic is meant onset of any of the various retinal changes described above or vision loss. In another embodiment, the composition is administered after disease becomes symptomatic. In yet another embodiment, the composition is administered after initiation of photoreceptor loss. In another embodiment, the composition is administered after outer nuclear layer (ONL) degeneration begins. In some embodiments, it is desirable that the composition is administered while bipolar cell circuitry to ganglion cells and optic nerve remains intact.

In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye. In another embodiment, the composition is administered to the entire eye.

In another embodiment, the method includes performing functional and imaging studies to determine the efficacy of the treatment. These studies include ERG and in vivo retinal imaging, as described in the examples below. In addition visual field studies, perimetry and microperimetry, pupillometry, mobility testing, visual acuity, contrast sensitivity, color vision testing may be performed.

In yet another embodiment, any of the above described methods is performed in combination with another, or secondary, therapy. The therapy may be any now known, or as yet unknown, therapy which helps prevent, arrest or ameliorate any of the described retinal changes and/or vision loss. In one embodiment, the secondary therapy is encapsulated cell therapy (such as that delivering Ciliary Neurotrophic Factor (CNTF)). See, e.g., Sieving, P. A. et al, 2006. Proc Natl Acad Sci USA, 103(10):3896-3901, which is hereby incorporated by reference. In another embodiment, the secondary therapy is a neurotrophic factor therapy (such as pigment epithelium-derived factor, PEDF; ciliary neurotrophic factor 3; rod-derived cone viability factor (RdCVF) or glial-derived neurotrophic factor). In another embodiment, the secondary therapy is anti-apoptosis therapy (such as that delivering X-linked inhibitor of apoptosis, XIAP). In yet another embodiment, the secondary therapy is rod-derived cone viability factor 2. The secondary therapy can be administered before, concurrent with, or after administration of any of the CFH polypeptides or biologically active fragments and/or variants thereof described above.

In some embodiments, any of the CFH polypeptides or biologically active fragments and/or variants thereof disclosed herein is administered to a subject in combination with another therapeutic agent or therapeutic procedure. In some embodiments, the additional therapeutic agent is an anti-VEGF therapeutic agent (e.g., an anti-VEGF antibody or fragment thereof such as ranibizumab, bevacizumab or a decoy protein such as aflibercept), a vitamin or mineral (e.g., vitamin C, vitamin E, lutein, zeaxanthin, zinc or copper), omega-3 fatty acids, and/or Visudyne™. In some embodiments, the other therapeutic procedure is a diet having reduced omega-6 fatty acids, laser surgery, laser photocoagulation, submacular surgery, retinal translocation, and/or photodynamic therapy. In some embodiments, the additional therapeutic agent is a vector (e.g., an AAV vector) encoding a CFH protein or biologically active fragment/variant thereof and/or a CFI protein or a biologically active fragment/variant thereof.

In some embodiments, any of the CFH polypeptides or biologically active fragments and/or variants thereof disclosed herein is administered to a subject in combination with an additional agent needed for processing and/or improving the function of the CFH polypeptides or biologically active fragments and/or variants thereof. For example, the CFH polypeptide or biologically active fragment and/or variant thereof may be administered with an antibody (or a vector encoding that antibody) that potentiates the activity of the administered CFH polypeptide or biologically active fragment and/or variant thereof and an endogenous CFH protein. Examples of such antibodies are found in WO2016/028150 or WO2019139481, which are each incorporated herein in their entirety.

### Kits

In some embodiments, any of the CFH polypeptides or biologically active fragments and/or variants thereof disclosed herein is assembled into a pharmaceutical or diagnostic or research kit to facilitate their use in therapeutic, diagnostic or research applications. A kit may include one or more containers housing any of the CFH polypeptides or biologically active fragments and/or variants thereof disclosed herein and instructions for use. In some embodiments, the kit includes instructions for administering any of the CFH polypeptides or biologically active fragments and/or variants thereof to a subject in whom it has been determined has one or more of any of the CFH mutations disclosed herein. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit.

### EXAMPLES

The disclosure now being generally described, it will be more readily understood by reference to the following examples, which are included merely for purposes of illustration of certain embodiments and embodiments of the present disclosure, and are not intended to limit the disclosure.

### Example 1: CFH Mutant Analysis

Various CFH mutants were generated and tested in different functional assays. Assays are discussed below.

### Fluorescent CFI Cofactor Assay

Recombinant human CFH variants were serially diluted in Tris-buffered saline (1×TBS; 50 mM Tris, 150 mM NaCl, pH 7.4) at concentrations ranging from 0.006 to 0.5 mg/mL in a twelve point dose response. Assay components were added to opaque half-area black polystyrene plates in the following order in a 50 μL final reaction volume: 0.02 mg purified human C3b, 5 μM ANS, CFH in final concentrations from 1.2 to 100 μg/mL and 0.1 μg of CFI. Reactions were mixed briefly by shaking at 4000 revolutions per minute and read over 30 minutes at 30 seconds intervals at 30° C. Fluorescence readings were recorded in kinetic mode with excitation set to 386 nm and emission set to 472 nm. Negative controls for reaction rate included no C3b, no CFI and no CFH. All reagents, instruments, and laboratory supplies used in this experiment are listed in Table 1 and 2.

TABLE 1
Equipment and supplies

| Equipment | Manufacturer |
|---|---|
| SpectraMax M5e | Molecular Devices |
| Pipette (2 μL) | VistaLab |
| Pipette (20 μL) | VistaLab |
| Pipette (200 μL) | VistaLab |
| Pipette (1000 μL) | VistaLab |
| Plate shaker | Lab-Line Instruments |
| 96 well half area opaque polystyrene plates | Costar |
| Prism 7 for Windows | GraphPad |

TABLE 2
Reagents

| Reagent | Manufacturer | Concentration | Storage |
|---|---|---|---|
| 8-anilinonaphthalene- | Arcos | 100 mM in methyl | −20° C. |
| 1 sulfonic acid, CAS# | | sulfoxide; dilute 1:2000 | |
| 82-76-8 | | with 1X TBS | |
| Methyl sulfoxide, | Arcos | n/a | ambient |
| molecular biology grade; | | | |
| Cas#67-68-5 | | | |
| 10X Tris-buffered saline | Boston | Dilute to 1X with water | ambient |
| (TBS); 500 mM Tris, | Biomedical | | |
| 1.5M NaCl, pH7.4 | | | |
| Complement factor 3b | Complement | 1.03 mg/mL | −80° C. |
| | Technology | | |
| Complement factor 1 | Complement | 1.04 mg/mL; dilute to | −80° C. |
| | Technology | 0.01 mg/mL in 1X TBS | |
| rhCFH variant | Thermo | Various concentrations; | 4° C. |
| (155 kDa) | | dilute with 1X TBS | |
| | | between range 0.006 | |
| | | mg/mL to 0.5 mg/mL | |

Abbreviations:
kDa, kilodaltons

The kinetic plots were analyzed by assessment of the slope in the linear range (typically between 200 and 900 seconds). The reaction rates, i.e., the slopes of observed reduction in fluorescence at 472 nm (corresponding to C3b cleavage), were calculated for each CFH concentration, carried out in triplicate. The inverse of the average reaction rate plus or minus the standard deviation were plotted for each concentration of CFH in μg/ml on a log scale. The data were fit with a 4-point sigmoidal curve to calculate the EC50 value. The ratio of the EC50 for each CFH Variant over the EC50 for VYE was calculated.

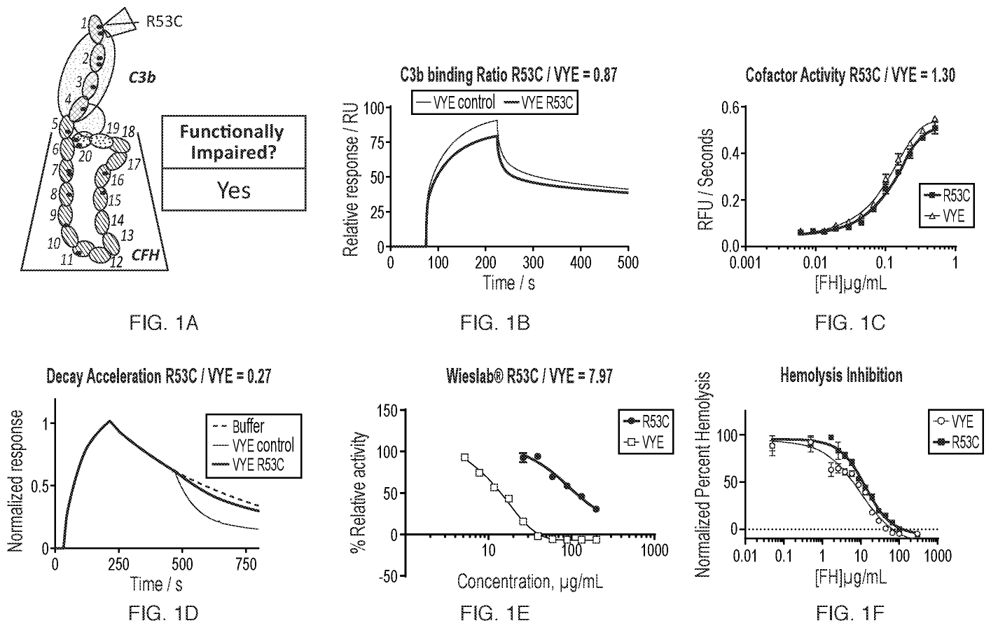

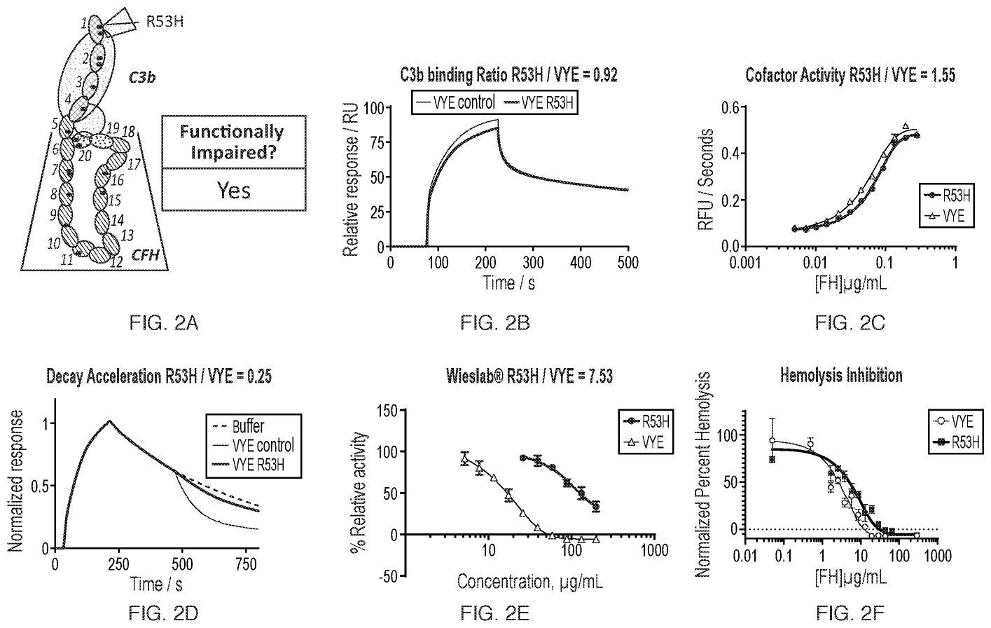

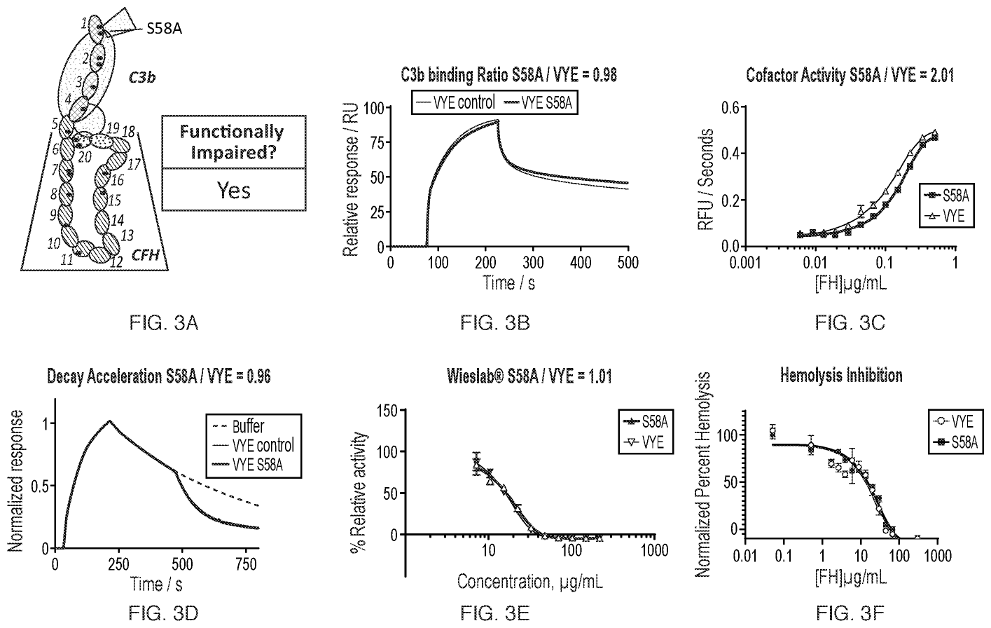

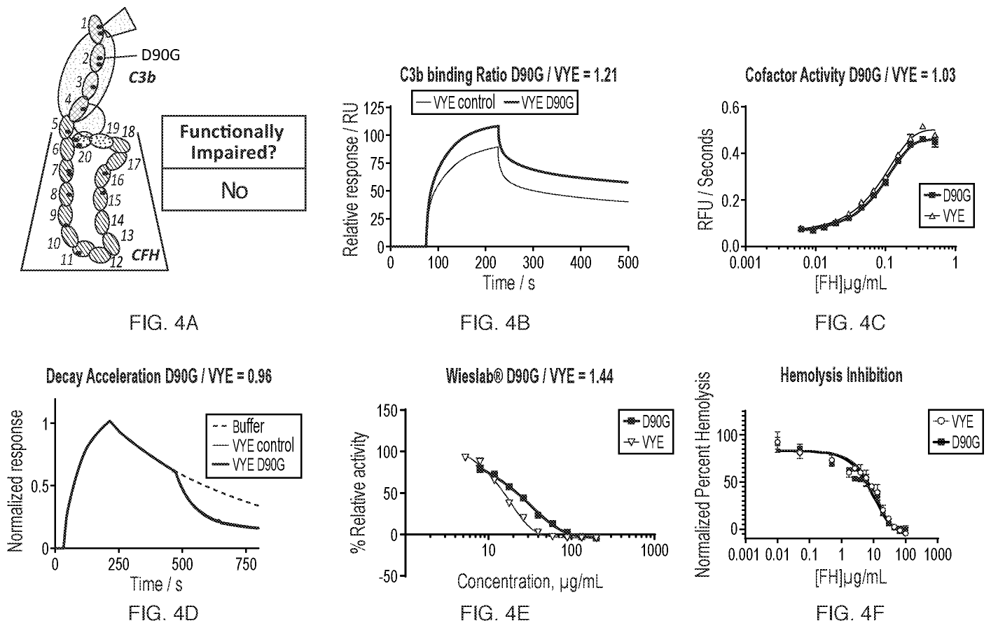

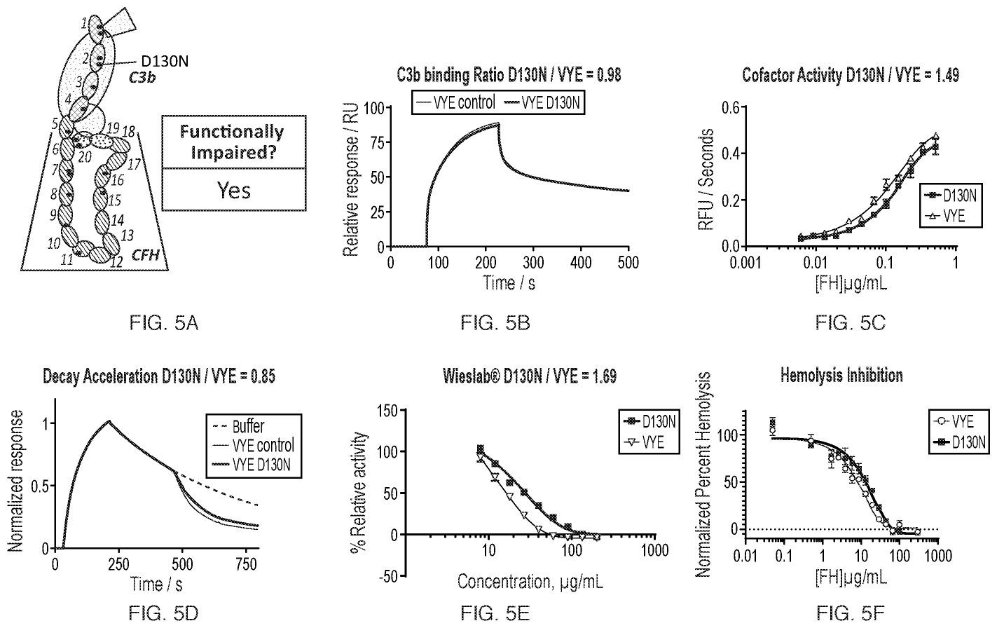

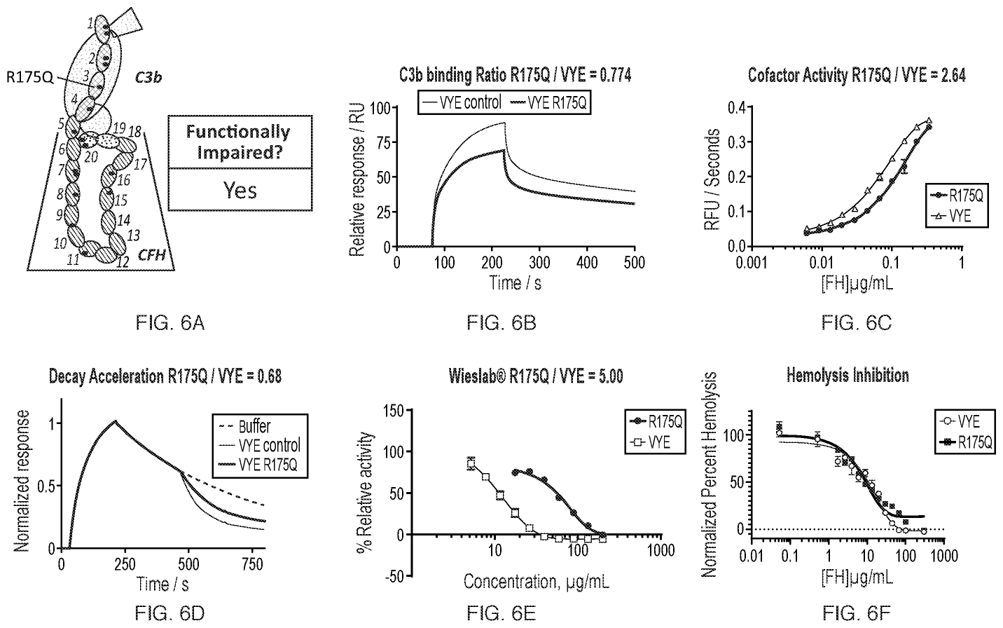

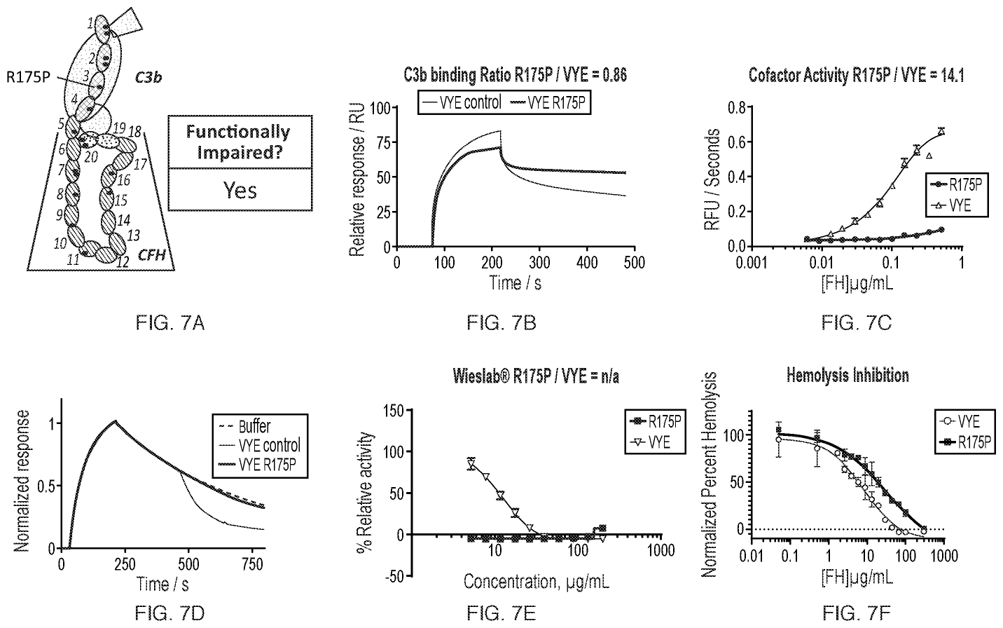

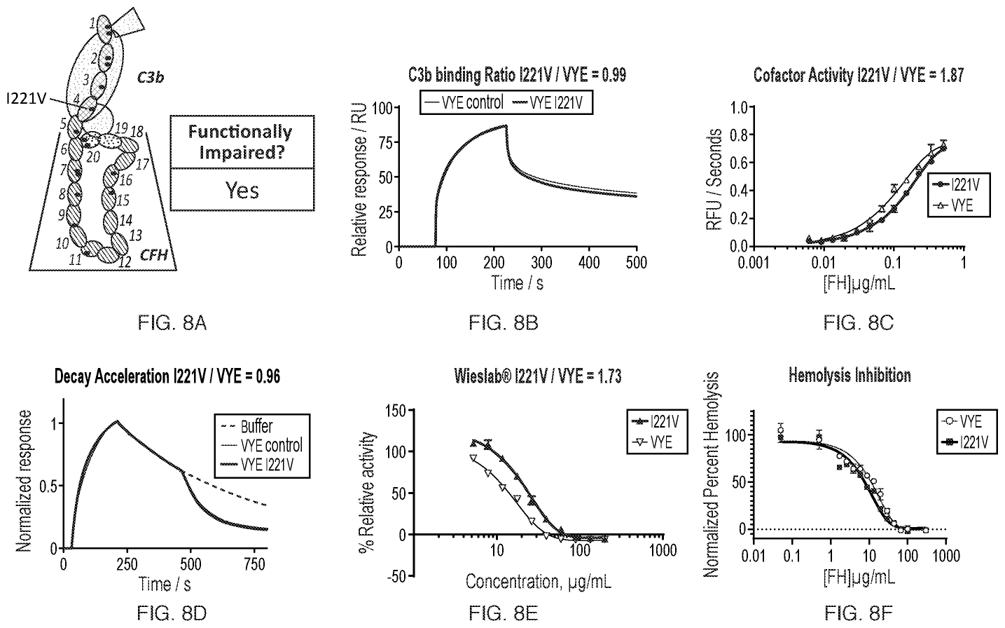

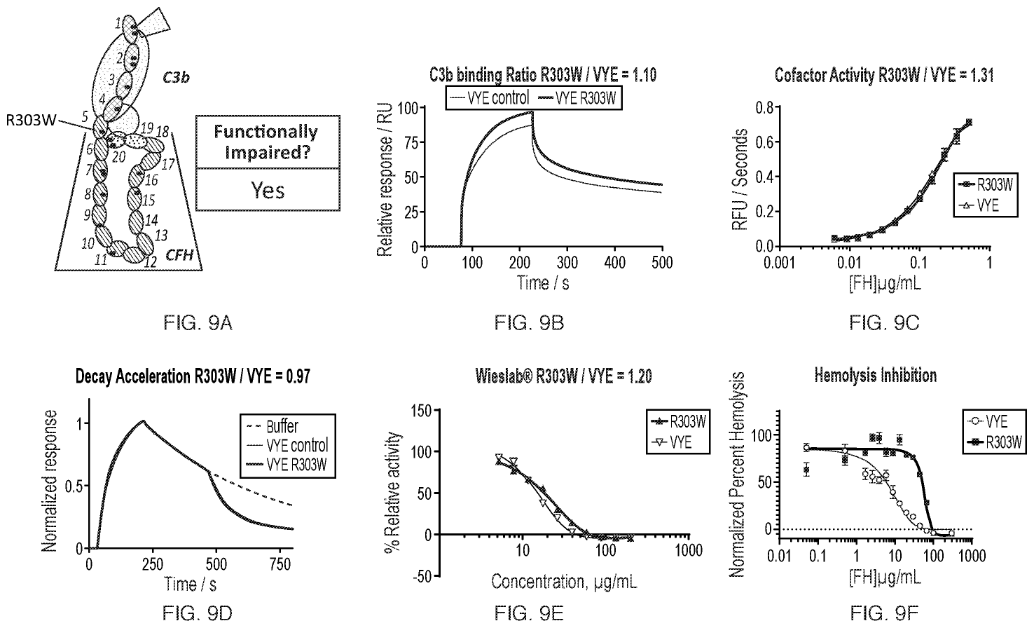

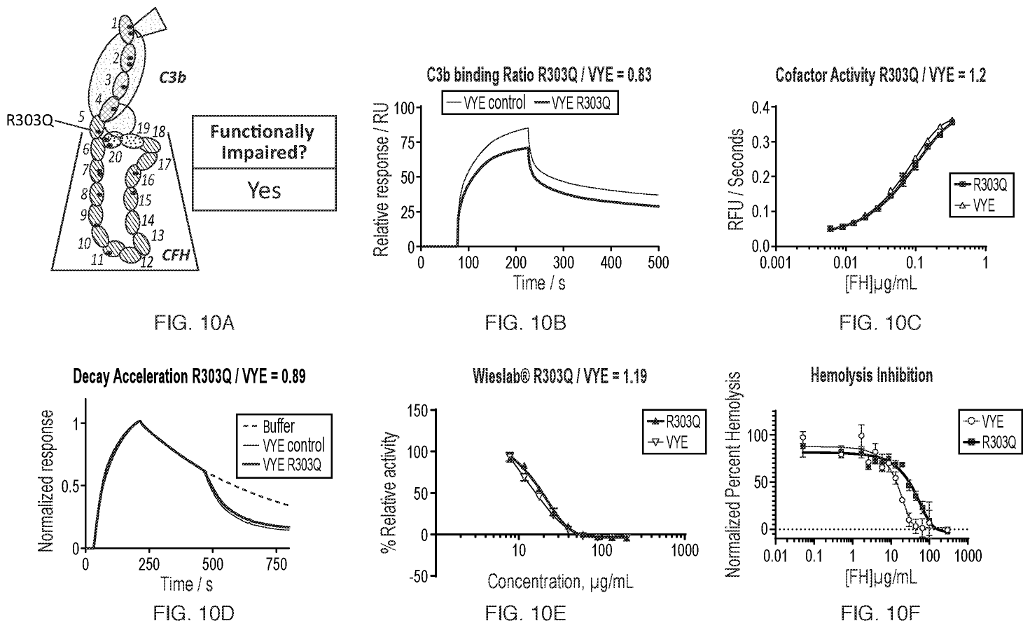

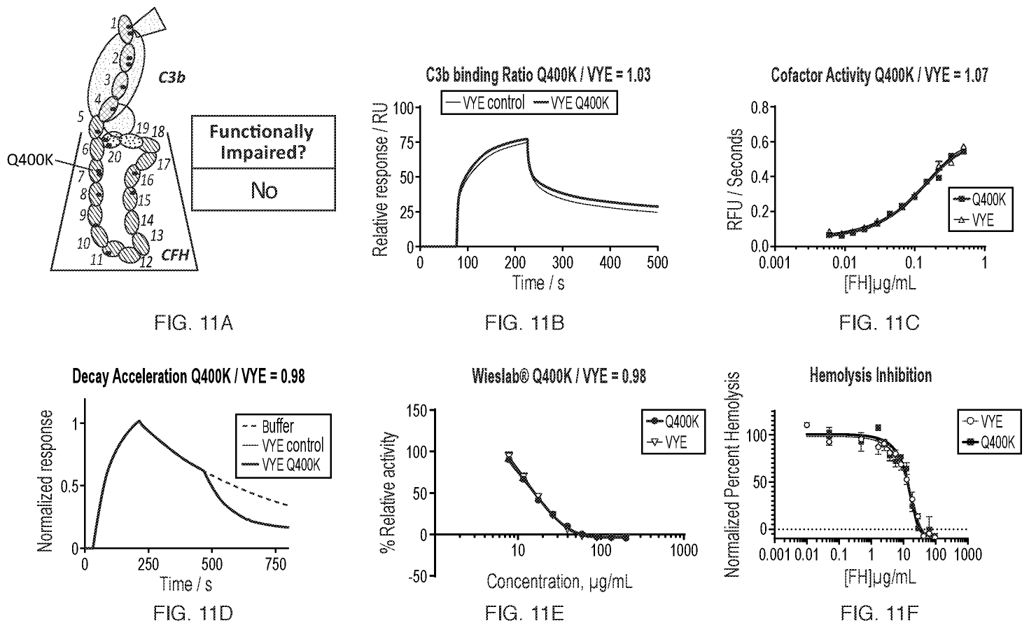

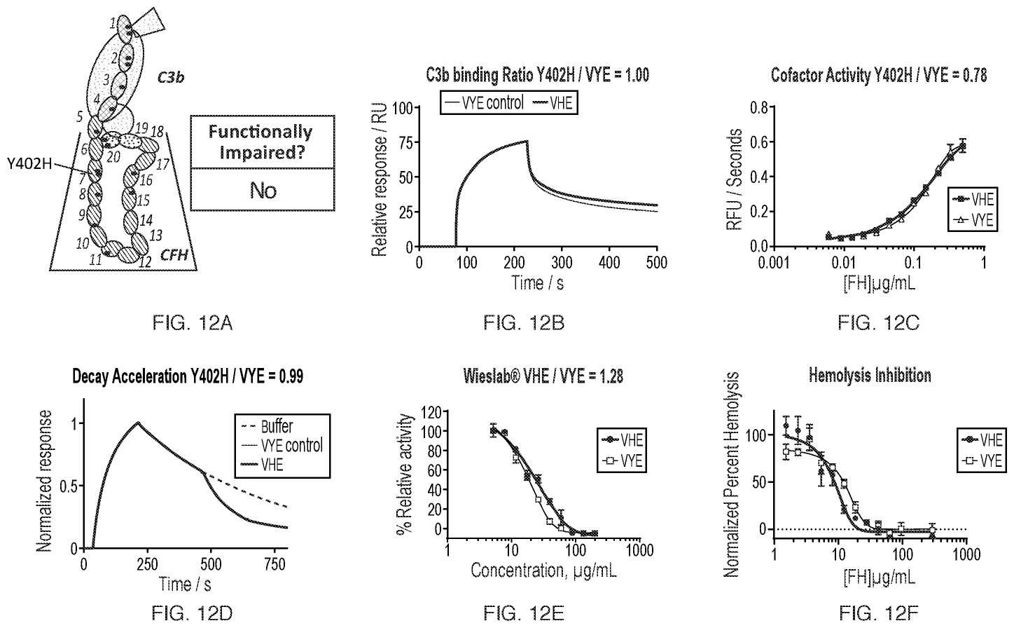

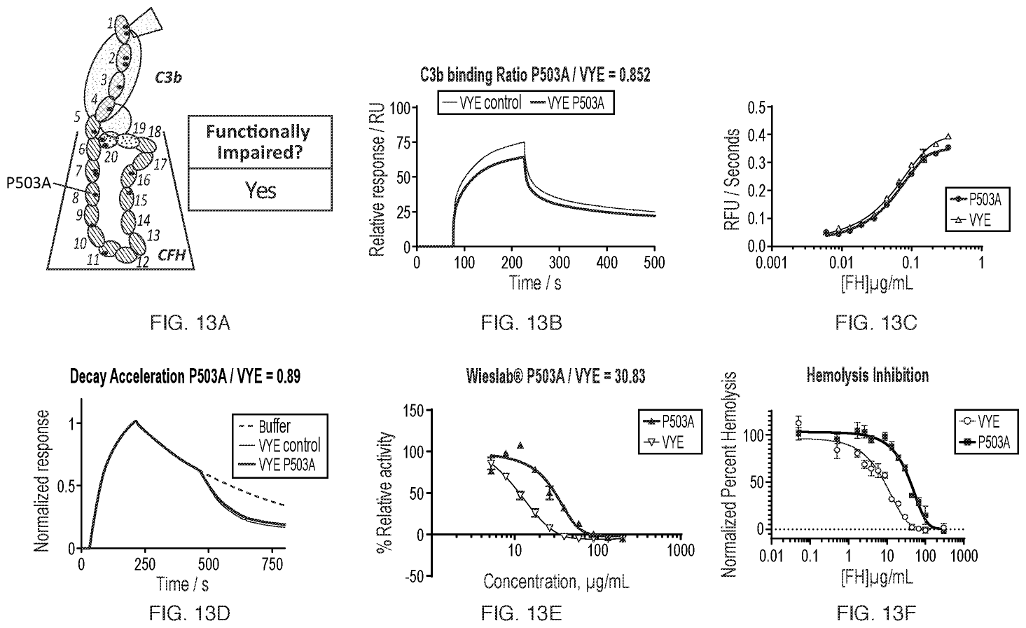

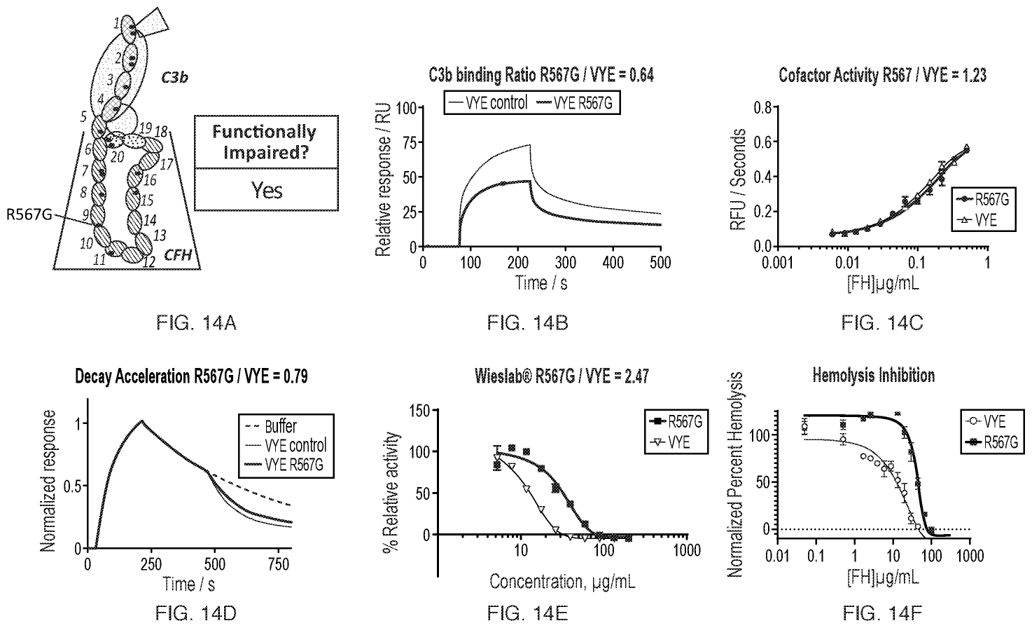

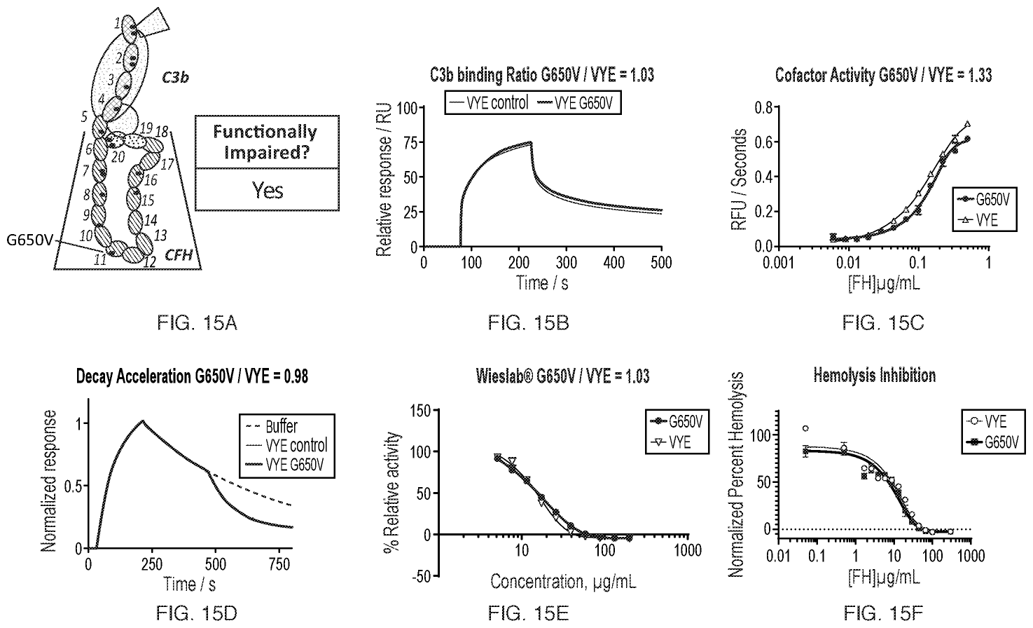

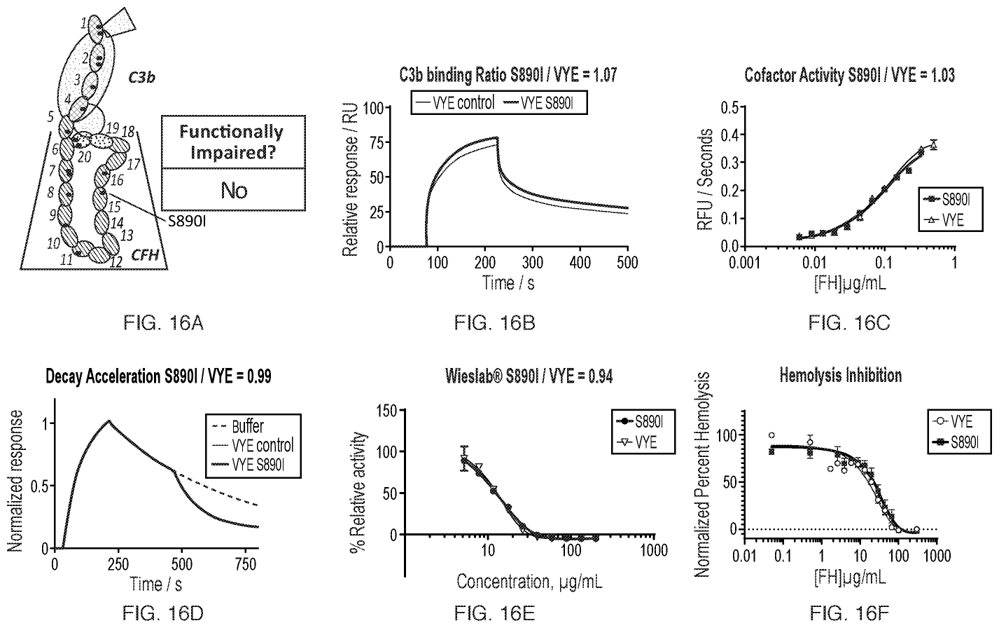

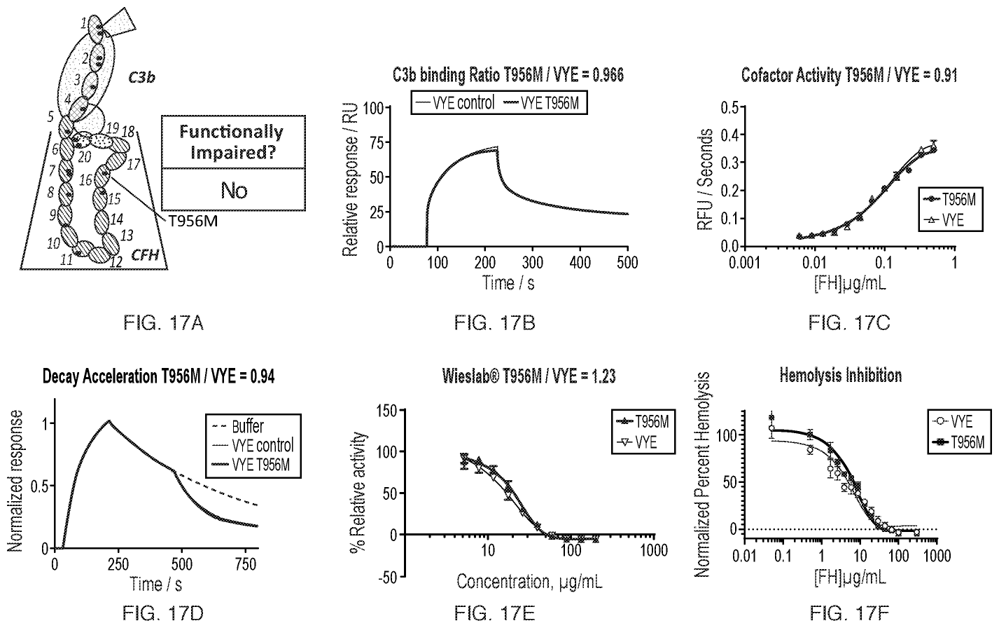

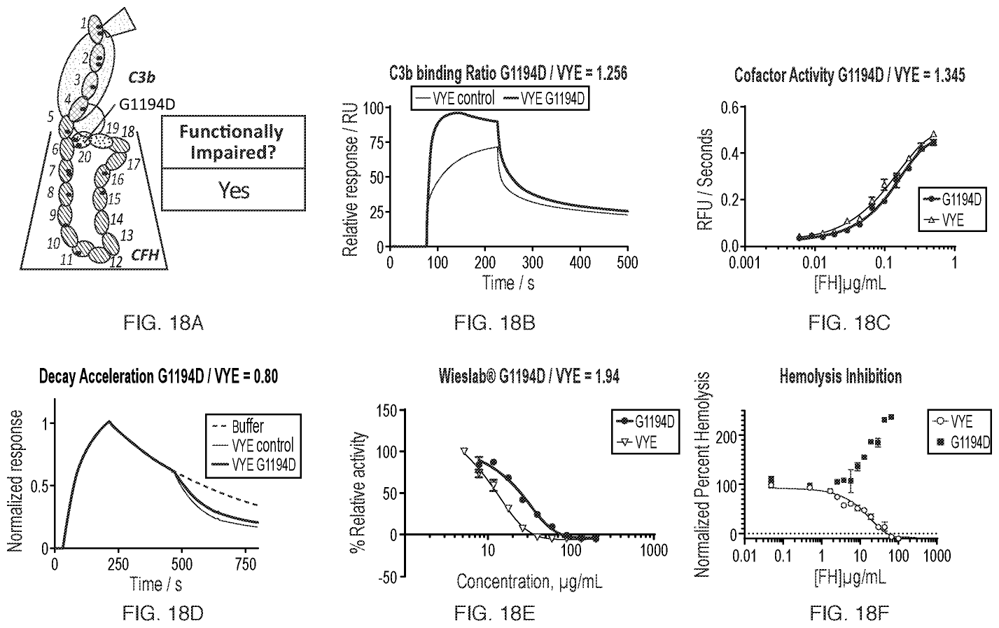

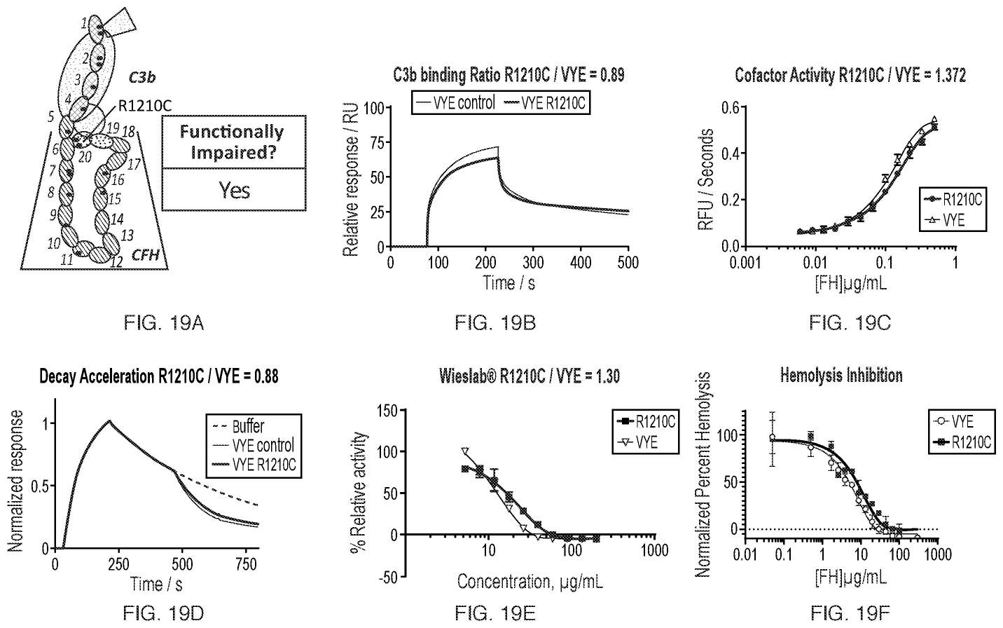

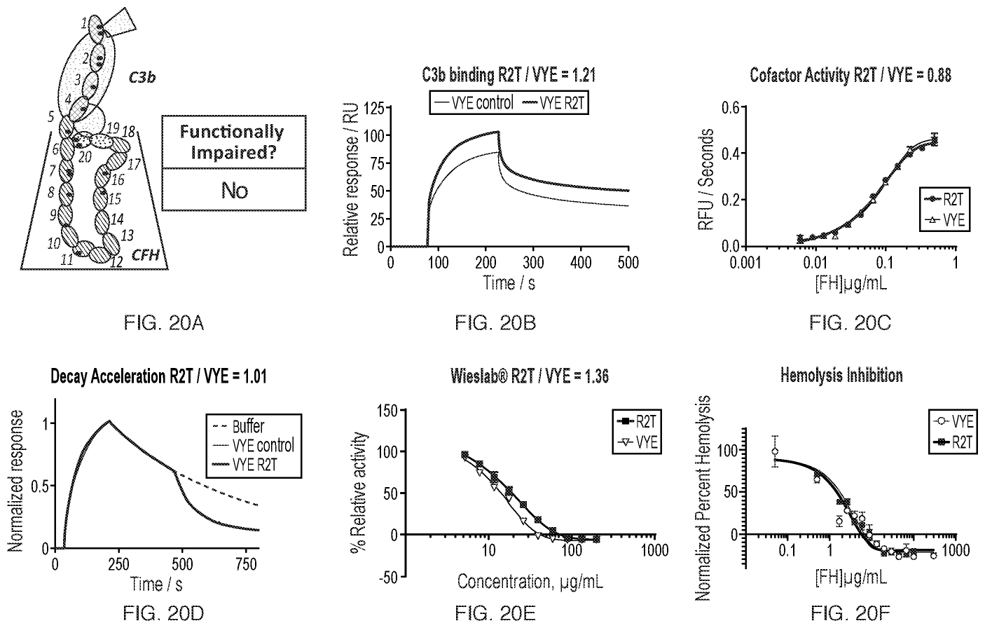

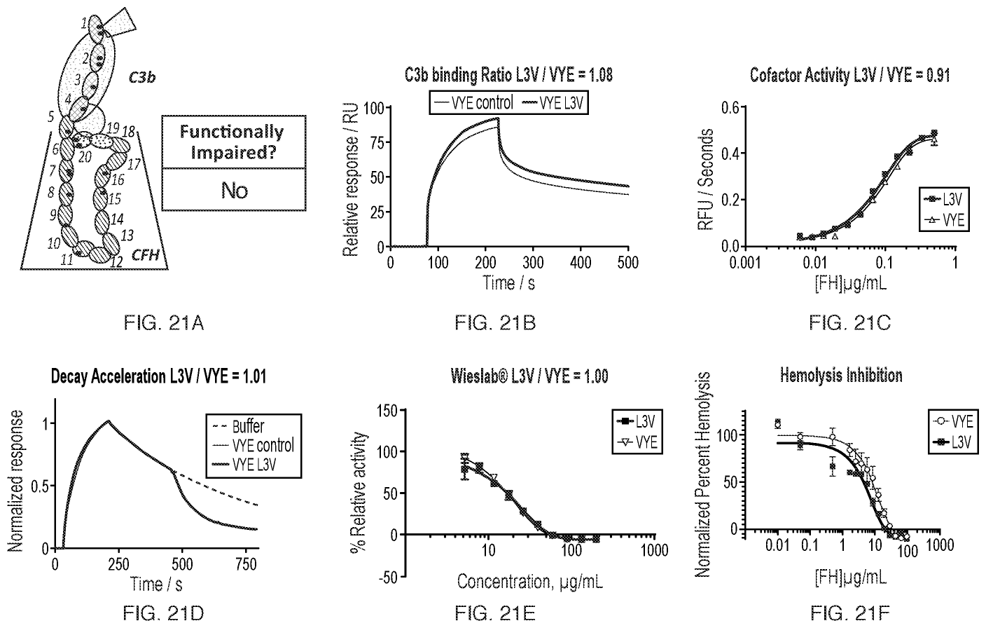

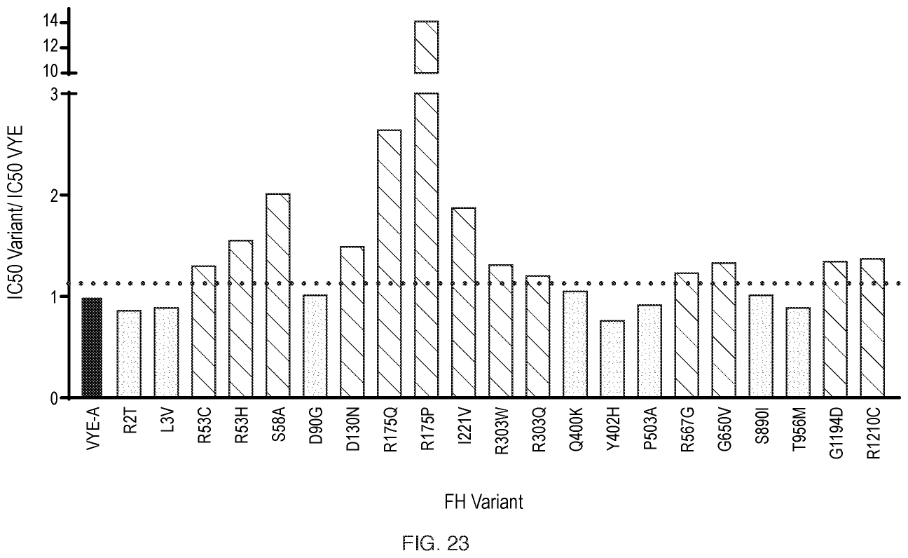

The inverse reaction rates (corresponding to C3b cleavage), at each concentration of CFH were plotted on log scale and fit to calculate the EC50 value. See FIGS. 1C, 2C, 3C, 4C, 5C, 6C, 7C, 8C, 9C, 10C, 11C, 12C, 13C, 14C, 15C, 16C, 17C, 18C, 19C, 20C, 21C. The ratio of the EC50 for each CFH Variant over the EC50 for VYE was calculated. A variant with a ratio >1.2 was considered to be functionally impaired compared to VYE. See FIG. 23.

### Hemolysis Assay

Human CFH-depleted serum (20 μL) was preincubated with a serially diluted set of concentrations of rhCFH variants ranging from 0.5 to 300 μg/mL (CFH concentrations were relative to the volume of serum) in gelatin-containing veronal buffer (GVB° with 0.1 mM EDTA) at room temperature for 10 minutes. SEs were washed once in GVB° without EDTA and resuspended to 2.1×10⁸ cells/mL in GVB° with 0.1 mM EDTA. A total of 2.1×10⁷ SE were added to each well followed by Mg EGTA at a final concentration of 10 mM in a total reaction volume of 200 μL. Wells containing reaction mixes in heat-inactivated, CFH-depleted serum and 1% triton-X in water were included as a negative and positive control for lysis, respectively. Samples were incubated at 37° C. for 60 minutes with shaking, followed by the addition of 150 μL of GVBE to stop further cell lysis. The samples were centrifuged at 1000×g for 5 minutes at 7° C. A volume of 150 μL of precleared supernatant was transferred to a clear bottom 96-well plate and the extent of hemolysis was measured in triplicate samples for each CFH concentration as absorbance at 412 nm (maximum absorbance for hemoglobin), corrected for background absorbance measured at 690 nm. All reagents, instruments and laboratory supplies used in this experiment are listed in Tables 3 and 4.

TABLE 3
Equipment and supplies

| Equipment | Manufacturer |
|---|---|
| SpectraMax M5e | Molecular Devices |
| Pipette (2 μL) | VistaLab |
| Pipette (20 μL) | VistaLab |
| Pipette (200 μL) | VistaLab |
| Pipette (1000 μL) | VistaLab |
| Multichannel pipette (20 μL) | VistaLab |
| Multichannel pipette (200 μL) | VistaLab |
| Shaking incubator | New Brunswick Scientific |
| 96 well deep well v-bottom plates | Costar |
| 96 well clear plates | Corning |
| Plate sealers | Thermo |
| Refrigerated centrifuge | Beckman |
| Water bath | Thermo |
| Prism 7 for Windows | GraphPad |

TABLE 4
Reagents

| Reagent | Manufacturer | Concentration | Storage |
|---|---|---|---|
| Gelatin-containing | Complement | 0.1% gelatin, 5 mM | 4° C. |
| veronal buffer (GVB°) | Technology | Veronal, 145 mM | |
| | | NaCl, 0.025% NaN3, | |
| | | pH 7.3 | |
| Gelatin-containing | Complement | 0.1% gelatin, 5 mM | 4° C. |
| veronal buffer with 10 | Technology | Veronal, 145 mM | |
| mM EDTA (GVBE) | | NaCl, 0.025% NaN3, | |
| | | 10 mM EDTA | |
| | | pH 7.3 | |
| Triton-X | Biovision | 10% in water | 4° C. |
| Mg•EGTA | Complement | 0.1M Mg•EGTA, pH | 4° C. |
| | Technology | 7.3 | |
| CFH-depleted human | Complement | n/a | −80° C. |
| serum | Technology | | |
| Sheep erythrocytes | Complement | 5 × 10⁸ cells/mL | 4° C. |
| | Technology | | |
| rhCFH (155 kDa) | Thermo | Various concentrations; | 4° C. |
| | | dilute with 1X TBS | |
| | | between 0.0005 mg/mL | |
| | | to 0.3 mg/mL | |

Abbreviations:
EDTA, Ethylenediaminetetraacetic acid;
EGTA, ethylene glycol-bis(β-aminoethyl ether)-N,N,N′,N′-tetraacetic acid;
kDa, kilodaltons

Corrected absorbance measurements were expressed as percentage of the 100% lysis control (CFH-depleted serum alone). The baseline hemolysis level was set using SEs incubated with heat-inactivated, CFH-depleted serum. The formula used to normalize each measurement to percent hemolysis is shown in Formula 1.

$$Formula 1 : Normalized percent hemolysis formula \\ normalized percent hemolysis = \frac{Xi - min ( X )}{max ( X ) - min ( X )} \times 100 ,$$

Xi is the individual measured value for each sample, max (X) is average of the values from triplicate samples incubated with CFH-depleted serum and min (X) is the average of the values from triplicate samples incubated with heat-inactivated CFH-depleted serum

For each concentration of CFH variant in μg/mL (on a log scale), the averaged normalized percent hemolysis values plus or minus the standard deviation were plotted. The data were fit with a 4-point sigmoidal curve from which the EC50 value was interpolated. Given the inherent variability of this assay, a numerical basis for defining functional impairment was not possible. Variants with a functional impairment were defined for those with an unequivocal difference in potency in the assay compared to VYE or, in cases where the effects were subtle, a trend towards less activity than VYE in at least 2 of 3 independent runs of the assay was used as a basis of defining functional impairment.

The extent of hemolysis was measured in CFH-depleted serum as well as serum enriched with each CFH variant. The average normalized percent hemolysis values plus or minus the standard deviation is plotted. The data were fit to interpolate the EC50 value. See FIGS. 1F, 2F, 3F, 4F, 5F, 6F, 7F, 8F, 9F, 10F, 11F, 12F, 13F, 14F, 15F, 16F, 17F, 18F, 19F, 20F, 21F.

### C3b Binding Affinity by SPR

SPR experiments were performed at 25° C. on a Biacore T200 instrument (GE Healthcare). A total of 300 RUs of C3b (supplied by Complement Technology, Inc., lot number 26a) were attached using standard amine coupling to one flow cell of a Biacore CM1 chip (Lot number: 10260563) (GE Healthcare). A second flow cell of the CM1 chip was used for background subtraction.

A 1 μM solution of each CFH variant, in 10 mM 4-(2-hydroxyethyl)-1-piperazineethanesulfonic acid (HEPES) buffer, 150 nM NaCl, 0.05% (v/v) surfactant P20, (pH 7.4) (GE Healthcare), was injected over amine-coupled C3b for 150 seconds at 50 !IL/minute followed by a dissociation time of 600 seconds. The sensor chip surface was regenerated between individual injections by three 30-second 50-μL/minute injections of 1 M NaCl, followed by a surface stabilization period of 150 seconds after the third NaCl injection. The baseline drift was corrected by subtracting the signal obtained from an injection of 0 μM CFH. Data were analyzed using the Biacore Evaluation software and a 1:1 steady-state binding model. For comparison, an identically prepared solution of VYE was flowed over the same flow cell. The corrected responses were then plotted against time, and the resultant plots overlaid at the time point of VYE/variant injection. The ratio C3b binding for each variant was compared to VYE and variants with ratios below 0.90 in duplicate independent experiments were considered to be functionally impaired for this activity. All reagents, instruments, and laboratory supplies used in this experiment are listed in Tables 5 and 6.

TABLE 5
Equipment and supplies

| Equipment | Manufacturer |
|---|---|
| Biacore T200 | GE Healthcare |
| Pipette (10 μL) ErgoOne ® | STARLAB |
| Pipette (100 μL) ErgoOne ® | STARLAB |
| Pipette (200 μL) ErgoOne ® | STARLAB |
| Pipette (1000 μL) ErgoOne ® | STARLAB |
| Biacore C1 chip | GE Healthcare |
| Slide-A-Lyzer ® Mini Dialysis | Thermo Scientific |
| Units | |
| Tube-O-Dialyzer ™ Medi | G-Biosciences |
| Biacore Evaluation software | GE Healthcare |
| Prism 7 for Windows | GraphPad |

TABLE 6
Reagents

| Reagent | Manufacturer | Concentration | Storage |
|---|---|---|---|
| 10 × HBS-P+ | GE Healthcare | Diluted to 1×, i.e., 10 | ambient |
| | | mM HEPES buffer, 150 | |
| | | nM NaCl, 0.05% (v/v) | |
| | | surfactant P20, (pH 7.4) | |
| Complement | Complement | 1.03 mg/mL | −80° C. |
| component 3b | Technology, | | |
| | Inc. | | |
| rhCFH variants | Thermo | Various concentrations; | 4° C. |
| (155 kDa) | | diluted with 1 × HBS- | |
| | | P+, to 1 μM | |

Abbreviations:
HBS-P+, HEPES-buffered saline with P20;
kDa, kilodaltons

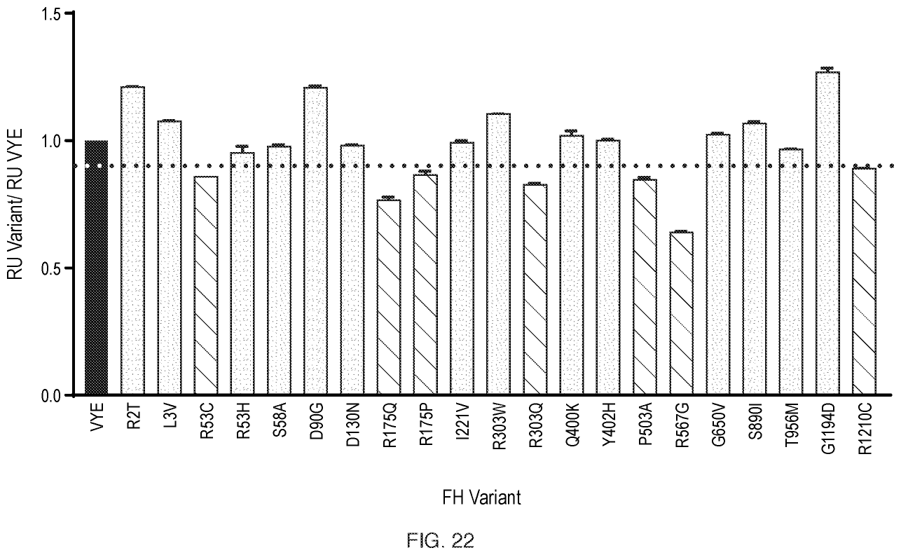

The C3b binding activity for each CFH variant was compared to the VYE control to determine the C3b binding ratio. See FIGS. 1B, 2B, 3B, 4B, 5B, 6B, 7B, 8B, 9B, 10B, 11B, 12B, 13B, 14B, 15B, 16B, 17B, 18B, 19B, 20B, 21B. Variants with ratios below 0.90 were considered functionally impaired for C3b binding activity. See FIG. 22.

### DAA Assay

The SPR experiments were performed at 25° C. on a Biacore T200 instrument (GE Healthcare) using 10 mM 4-(2-hydroxyethyl)-1-piperazineethanesulfonic acid (HEPES), 150 mM NaCl, 0.05% (v/v) surfactant P20, 1 mM MgCl2 (pH 7.4) (GE Healthcare). A total of 2845 response units (RUs) of C3b (lot number 26a) was immobilized to a flow cell of a Biacore CMS-sensor chip (Lot number: 10260535) using standard amine coupling. A reference flow cell was used for background subtraction. C3bBb was assembled on the CMS chip bearing immobilized C3b molecules, by performing a 180 second 10-μL/minute injection of a solution containing 500 nM CFB (lot number 17b) and 50 nM CFD (lot number 38). The decay of C3bBb was monitored over an initial 240 second dissociation phase in the absence of any CFH, allowing observation of intrinsic convertase decay (i.e., a decline in RUs attributable to loss of Bb). Subsequently, a 20nM solution of either VYE or a CFH variant were injected at 10 μL/minute for 180 seconds, allowing observation of accelerated dissociation. All reagents, instruments, and laboratory supplies used in this experiment are listed in Table 7 and 8.

TABLE 7
Equipment and supplies

| Equipment | Manufacturer |
|---|---|
| Biacore T200 | GE Healthcare |
| Pipette (10 μL) | STARLAB |
| Pipette (100 μL) | STARLAB |
| Pipette (200 μL) | STARLAB |
| Pipette (1000 μL) | STARLAB |
| Biacore CM5 chip | GE Healthcare |
| Slide-A-Lyzer ® Mini | Thermo Scientific |
| Dialysis Units | |
| Tube-O-Dialyzer ™ Medi | G-Biosciences |
| Biacore Evaluation software | GE Healthcare |
| Prism 7 for Windows | GraphPad |

TABLE 8
Reagents

| Reagent | Manufacturer | Concentration | Storage |
|---|---|---|---|
| 10 × HBS-P+ | GE Healthcare | Diluted to 1×, i.e., 10 mM | ambient |
| | | HEPES buffer, 150 nM NaCl, | |
| | | 0.05% (v/v) surfactant P20, | |
| | | and supplemented with 1 mM | |
| | | MgCl₂ (pH 7.4) | |
| Complement | Complement | 1.0 mg/mL | −80° C. |
| factor B | Technology, Inc. | | |
| Complement | Complement | 0.1 mg/mL | −80° C. |
| factor D | Technology, Inc. | | |
| Complement | Complement | 1.03 mg/mL | −80° C. |
| component | Technology, Inc. | | |
| 3b | | | |
| rhCFH | Thermo | Various concentrations; | 4° C. |
| variants | | diluted with 1 × HBS-P+, | |
| | | 1 mM MgCl₂, to 20 nM | |

The SPR response arising from the binding of each CFH variant to immobilized C3b, in the absence of the convertase, was subtracted from the corresponding convertase decay response. Data were processed using the Biacore Evaluation software. Experimental data were then normalized to compensate for the small drift in signal (assumed to arise from the gradual leaching of C3b from the surface over multiple measurements). Normalization of data was achieved by comparing responses at 220 seconds (i.e., at the time when the injection of CFB and CFD ceased) and adjusting these to be 1.0. The normalized responses were then plotted against time, and the resultant plots overlaid at the time point of VYE/variant injection.

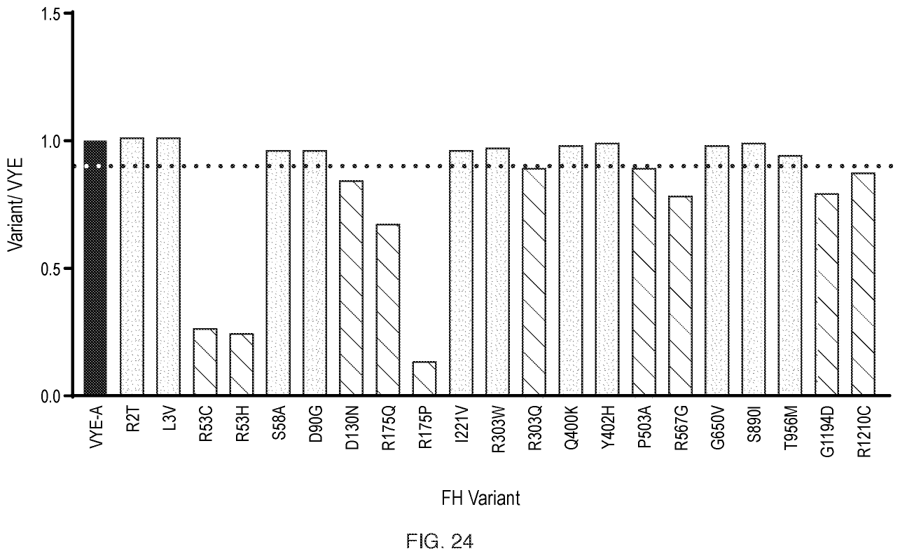

Decay of the surface plasmon resonance (SPR) response of C3 convertase (C3bBb) dissociation of Bb from C3b. Addition of VYE accelerates the dissociation of the C3 convertase and the decay acceleration of each CFH variant was compared to that of VYE. Normalized responses are plotted against time. See FIGS. 1D, 2D, 3D, 4D, 5D, 6D, 7D, 8D, 9D, 10D, 11D, 12D, 13D, 14D, 15D, 16D, 17D, 18D, 19D, 20D, 21D. The ratios of DAA for each variant were compared to VYE and variants with ratios below 0.85 were considered to be functionally impaired for this activity. See FIG. 24.

### Weislab Assay

The Wieslab kit (Cat #COMPLAP330) was used as directed with the following modifications. Instead of NHS, human serum depleted of CFH (Complement Technology; A337; Lot #11) that was supplemented with either VYE or a CFH variant in a dose response from 5.2 to 200 ug/ml was utilized with the kit components. All other kit reagents were used as directed. The data for each variant were normalized to the activity of CFH depleted serum alone and the relative activity was plotted against CFH concentration in ug/ml. The ratio of each variant's interpolated IC50 over that for VYE was calculated and values greater than 1.6 were used to determine functional impairment.

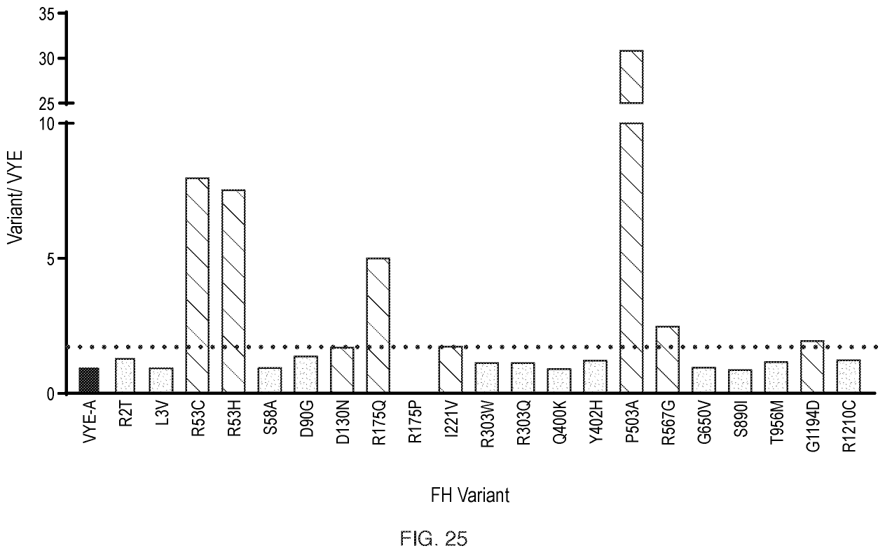

The Wieslab® kit (Svar Life Science AB) was used to determine the percent activity of serum enriched with each CFH variant normalized to the activity of CFH depleted serum alone. The relative activity was plotted against CFH concentration in μg/ml. The ratio of each variant's interpolated IC50 over that for VYE was calculated and CFH variants having values greater than 1.6 were considered functionally impaired. See FIGS. 1E, 2E, 3E, 4E, 5E, 6E, 7E, 8E, 9E, 10E, 11E, 12E, 13E, 14E, 15E, 16E, 17E, 18E, 19E, 20E, 21E. See also FIG. 25.

### Cellular Expression Assay

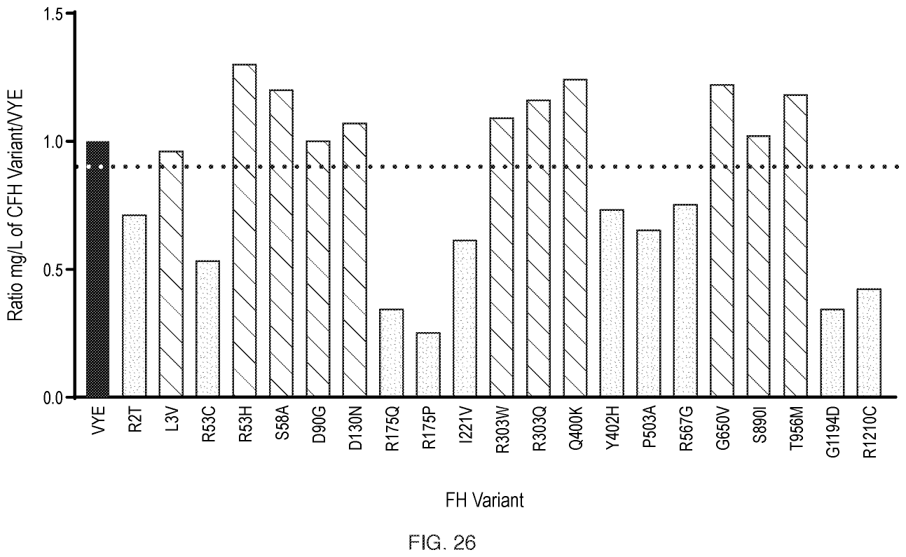

DNA plasmids encoding each of the variants were prepared and transfected transiently into cells. Cells were cultured for five days and the supernatants collected and cells removed. The CFH variant in each of the supernatants was then purified using a proprietary CFH affinity resin. Upon elution and pooling of fractions containing CFH the protein concentration was measured and the total protein recovered was calculated. For each variant the amount of protein produced per unit volume was compared to wild type CFH and the ratio calculated. The ratio of cellular expression of each CFH variant compared to expression of the CFH control was plotted. Functionally deficient CFH variants were determined to have a ratio<0.9. See FIG. 26.

### Summary

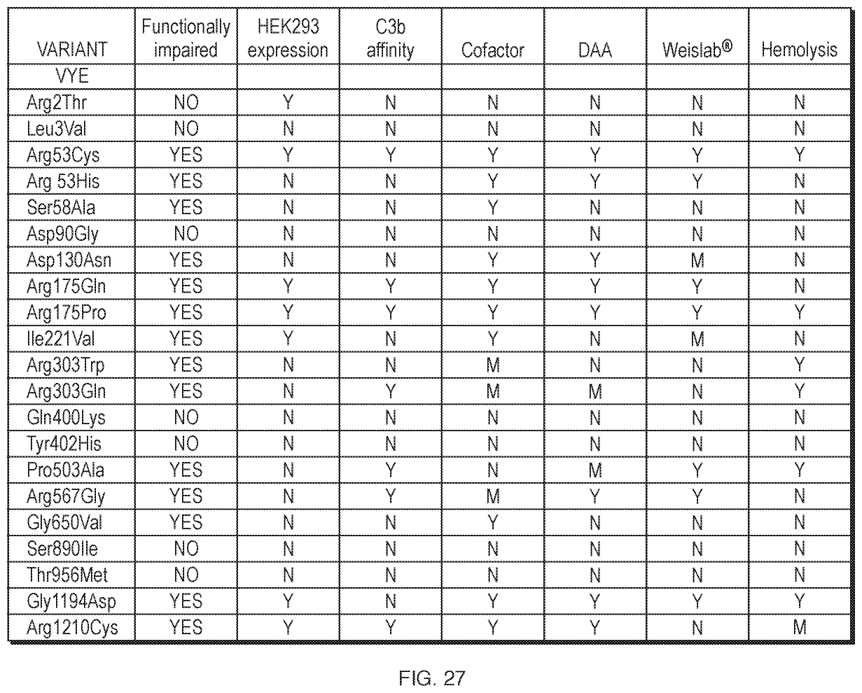

FIG. 27 shows summary functional assay data for each CFH variant tested as compared to VYE control CFH.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

### NUMBERED EMBODIMENTS

Embodiments disclosed herein include embodiments P1 to P98 as provided in the numbered embodiments of the disclosure.

Embodiment P1: A method of treating a subject having a disease or disorder associated with undesired activity of the alternative complement pathway, comprising the step of administering to the subject a complement factor H (CFH) polypeptide or biologically active fragment and/or variant thereof, wherein the subject has one or more CFH gene mutations.

Embodiment P2: A method of treating a subject having age-related macular degeneration (AMD), comprising the step of administering to the subject a complement factor H (CFH) polypeptide or biologically active fragment and/or variant thereof, wherein the subject has one or more CFH gene mutations.

Embodiment P3: The method of embodiment P1 or P2, wherein the CFH polypeptide biologically active fragment and/or variant thereof is capable of acting as a cofactor with CFI to facilitate C3b cleavage.

Embodiment P4: The method of any one of embodiments P1-P3, wherein the CFH polypeptide or biologically active fragment and/or variant thereof is capable of diffusing across the Bruch's membrane.

Embodiment P5: The method of any one of embodiments P1-P4, wherein the CFH polypeptide or biologically active fragment and/or variant thereof is capable of binding C3b.

Embodiment P6: The method of any one of embodiments P1-P5, wherein the CFH polypeptide or biologically active fragment and/or variant thereof is capable of facilitating the breakdown of C3b.

Embodiment P7: The method of any one of embodiments P1-P6, wherein the CFH polypeptide or biologically active fragment and/or variant thereof is capable of binding to a cell surface (e.g., an erythrocyte and/or endothelial cell).

Embodiment P8: The method of any one of embodiments P1-P7, wherein the CFH polypeptide or biologically active fragment and/or variant thereof is capable of binding to heparin.

Embodiment P9: The method of any one of embodiments P1-P8, wherein the CFH polypeptide or biologically active fragment and/or variant thereof is capable of reducing C5b9 levels generated as a result of complement activation (e.g., as measured in a Wieslab AP assay (see, e.g., Example 1).

Embodiment P10: The method of any one of embodiments P1-P9, wherein the CFH polypeptide or biologically active fragment and/or variant thereof is capable of inhibiting hemolysis.

Embodiment P11: The method of any one of embodiments P1-P10, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 1, or a biologically active fragment thereof.

Embodiment P12: The method of any one of embodiments P1-P10, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 2, or a biologically active fragment thereof.

Embodiment P13: The method of any one of embodiments P1-P10, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 3, or a biologically active fragment thereof.

Embodiment P14: The method of any one of embodiments P1-P10, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 4, or a biologically active fragment thereof.

Embodiment P15: The method of any one of embodiments P1-P10, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 5, or a biologically active fragment thereof.

Embodiment P16: The method of any one of embodiments P1-P10, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 6, or a biologically active fragment thereof.

Embodiment P17: The method of any one of embodiments P1-P10, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 7, or a biologically active fragment thereof.

Embodiment P18: The method of any one of embodiments P1-P10, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 8, or a biologically active fragment thereof.

Embodiment P19: The method of any one of embodiments P1-P10, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 9, or a biologically active fragment thereof.

Embodiment P20: The method of any one of embodiments P1-P19, wherein the CFH polypeptide or biologically active fragment and/or variant thereof is encoded by a nucleotide sequence that is at least 70%, 75%, 80%, 85%, 90%, 91%, 92%, 93%, 94%, 95%, 96%, 97%, 98%, 99% or 100% identical to the nucleotide sequence of any one of SEQ ID NOs: 10-13, or a biologically active fragment thereof.

Embodiment P21: The method of any one of embodiments P1-P20, wherein the CFH polypeptide or biologically active fragment and/or variant thereof is a mature CFH polypeptide or biologically active fragment and/or variant thereof.

Embodiment P22: The method of any one of embodiments Pl-P21, wherein the subject is a human.

Embodiment P23: The method of embodiment P22, wherein the human is at least 40 years of age.

Embodiment P24: The method of embodiment P22, wherein the human is at least 50 years of age.

Embodiment P25: The method of embodiment P22, wherein the human is at least 65 years of age.

Embodiment P26: The method of any one of embodiments P1-P25, wherein the CFH polypeptide or biologically active fragment and/or variant thereof is administered locally.

Embodiment P27: The method of any one of embodiments P1-P25, wherein the CFH polypeptide or biologically active fragment and/or variant thereof is administered systemically.

Embodiment P28: The method of any one of embodiments P1-P27, wherein the subject has a loss-of-function mutation in the subject's CFH gene.

Embodiment P29: The method of any one of embodiments P1-P27, wherein the subject has any one of the following CFH mutations: R2T, L3V, R53C, R53H, S58A, D90G, D130N, R175Q, R175P, I221V, R303W, R303Q, Q400K, Y402H, P503A, R567G, G650V, S890I, T956M, G1194D, or R1210C.

Embodiment P30: The method of any one of embodiments P1-P27, wherein the subject has any one or more of the following CFH mutations: R2T, R53C, R53H, S58A, D130N, R175Q, R175P, I221V, R303W, R303Q, P503A, R567G, G650V, G1194D, or R1210C.

Embodiment P31: The method of any one of embodiments P1-P27, wherein the subject has an R2T mutation.

Embodiment P32: The method of any one of embodiments P1-P27, wherein the subject has an L3V mutation.

Embodiment P33: The method of any one of embodiments P1-P27, wherein the subject has an R53C mutation.

Embodiment P34: The method of any one of embodiments P1-P27, wherein the subject has an R53H mutation.

Embodiment P35: The method of any one of embodiments P1-P27, wherein the subject has an S58A mutation.

Embodiment P36: The method of any one of embodiments P1-P27, wherein the subject has a D90G mutation.

Embodiment P37: The method of any one of embodiments P1-P27, wherein the subject has a D130N mutation.

Embodiment P38: The method of any one of embodiments P1-P27, wherein the subject has an R175Q mutation.

Embodiment P39: The method of any one of embodiments P1-P27, wherein the subject has an R175P mutation.

Embodiment P40: The method of any one of embodiments P1-P27, wherein the subject has an I221V mutation.

Embodiment P41: The method of any one of embodiments P1-P27, wherein the subject has an R303W mutation.

Embodiment P42: The method of any one of embodiments P1-P27, wherein the subject has an R303Q mutation.

Embodiment P43: The method of any one of embodiments P1-P27, wherein the subject has a Q400K mutation.

Embodiment P44: The method of any one of embodiments P1-P27, wherein the subject has a Y402H mutation.

Embodiment P45: The method of any one of embodiments P1-P27, wherein the subject has a P503A mutation.

Embodiment P46: The method of any one of embodiments P1-P27, wherein the subject has an R567G mutation.

Embodiment P47: The method of any one of embodiments P1-P27, wherein the subject has a G650V mutation.

Embodiment P48: The method of any one of embodiments P1-P27, wherein the subject has an S890I mutation.

Embodiment P49: The method of any one of embodiments P1-P27, wherein the subject has a T956M mutation.

Embodiment P50: The method of any one of embodiments P1-P27, wherein the subject has a G1194D mutation.

Embodiment P51: The method of any one of embodiments P1-P27, wherein the subject has an R1210C mutation.

Embodiment P52: The method of any one of embodiments P1-P45, wherein the subject expresses a mutant CFH polypeptide having reduced CFH activity as compared to a wildtype CFH polypeptide (e.g., a CFH polypeptide having the amino acid sequence of SEQ ID NO: 1, 2, or 3).

Embodiment P53: The method of embodiment P52, wherein the CFH activity is the ability to bind to C3b.

Embodiment P54: The method of embodiment P52, wherein the CFH activity has the ability to act as a cofactor with CFI and facilitate C3b cleavage.

Embodiment P55: The method of embodiment P52, wherein the CFH activity is the ability to bind to a cell surface (e.g., an erythrocyte and/or endothelial cell).

Embodiment P56: The method of embodiment P52, wherein the CFH activity is the ability to bind to heparin.

Embodiment P57: The method of embodiment P52, wherein the CFH activity is the ability to reduce C5b9 levels generated as a result of complement activation, e.g., as measured in a Wieslab AP assay.

Embodiment P58: The method of embodiment P52, wherein the CFH activity is the ability to inhibit hemolysis.

Embodiment P59: The method of any one of embodiments P1-P58, wherein if a CFH polypeptide having the CFH mutation were tested in a functional assay, the mutant CFH polypeptide would display reduced CFH activity as compared to a wildtype CFH polypeptide (e.g., a CFH polypeptide having the amino acid sequence of SEQ ID NO: 1, 2, or 3).

Embodiment P60: The method of embodiment P59, wherein the functional assay tests the ability of CFH to bind to C3b.

Embodiment P61: The method of embodiment P59, wherein the functional assay tests the ability of CFH to act as a cofactor with CFI and facilitate C3b cleavage.

Embodiment P62: The method of embodiment P59, wherein the functional assay tests the ability of CFH to bind to a cell surface (e.g., an erythrocyte and/or endothelial cell).

Embodiment P63: The method of embodiment P59, wherein the functional assay tests the ability of CFH to bind to heparin.

Embodiment P64: The method of embodiment P59, wherein the functional assay tests the ability of CFH to reduce C5b9 levels generated as a result of complement activation, e.g., as measured in a Wieslab AP assay.

Embodiment P65: The method of embodiment P59, wherein the functional assay tests the ability of CFH to inhibit hemolysis.

Embodiment P66: The method of any one of embodiments P1-P65, wherein the subject has atypical hemolytic uremic syndrome (aHUS).

Embodiment P67: The method of any one of embodiments P1-P66, wherein the subject is suffering from a renal disease or complication.

Embodiment P68: The method of any one of embodiments P1-P67, wherein the subject is a subject in whom it has been determined has one or more CFH mutations.

Embodiment P69: The method of embodiment P68, wherein the subject is a subject in whom it has been determined has any one of the following CFH mutations: R2T, L3V, R53C, R53H, S58A, D90G, D130N, R175Q, R175P, I221V, R303W, R303Q, Q400K, Y402H, P503A, R567G, G650V, S890I, T956M, G1194D, or R1210C.

Embodiment P70: The method of embodiment P68, wherein the subject is a subject in whom it has been determined has any one or more of the following CFH mutations: R2T, R53C, R53H, S58A, D130N, R175Q, R175P, I221V, R303W, R303Q, P503A, R567G, G650V, G1194D, or R1210C.

Embodiment P71: The method of embodiment P68, wherein the subject is a subject in whom it has been determined has an R2T mutation.

Embodiment P72: The method of embodiment P68, wherein the subject is a subject in whom it has been determined has an L3V mutation.

Embodiment P73: The method of embodiment P68, wherein the subject is a subject in whom it has been determined has an R53C mutation.

Embodiment P74: The method of embodiment P68, wherein the subject is a subject in whom it has been determined has an R53H mutation.

Embodiment P75: The method of embodiment P68, wherein the subject is a subject in whom it has been determined has an S58A mutation.

Embodiment P76: The method of embodiment P68, wherein the subject is a subject in whom it has been determined has a D90G mutation.

Embodiment P77: The method of embodiment P68, wherein the subject is a subject in whom it has been determined has a D130N mutation.

Embodiment P78: The method of embodiment P68, wherein the subject is a subject in whom it has been determined has an R175Q mutation.

Embodiment P79: The method of embodiment P68, wherein the subject is a subject in whom it has been determined has an R175P mutation.

Embodiment P80: The method of embodiment P68, wherein the subject is a subject in whom it has been determined has an I221V mutation.

Embodiment P81: The method of embodiment P68, wherein the subject is a subject in whom it has been determined has an R303W mutation.

Embodiment P82: The method of embodiment P68, wherein the subject is a subject in whom it has been determined has an R303Q mutation.

Embodiment P83: The method of embodiment P68, wherein the subject is a subject in whom it has been determined has a Q400K mutation.

Embodiment P84: The method of embodiment P68, wherein the subject is a subject in whom it has been determined has a Y402H mutation.

Embodiment P85: The method of embodiment P68, wherein the subject is a subject in whom it has been determined has a P503A mutation.

Embodiment P86: The method of embodiment P68, wherein the subject is a subject in whom it has been determined has an R567G mutation.

Embodiment P87: The method of embodiment P68, wherein the subject is a subject in whom it has been determined has a G650V mutation.

Embodiment P88: The method of embodiment P68, wherein the subject is a subject in whom it has been determined has an S8901 mutation.

Embodiment P89: The method of embodiment P68, wherein the subject is a subject in whom it has been determined has a T956M mutation.

Embodiment P90: The method of embodiment P68, wherein the subject is a subject in whom it has been determined has a G1194D mutation.

Embodiment P91: The method of embodiment P68, wherein the subject is a subject in whom it has been determined has a R1210C mutation.

Embodiment P92: The method of any one of embodiments P68-P90, wherein the subject is a subject in whom it has been determined is homozygous for at least one of the one or more CFH mutations.

Embodiment P93: The method of any one of embodiments P68-P90, wherein the subject is a subject in whom it has been determined is heterozygous for at least one of the one or more CFH mutations.

Embodiment P94: The method of any one of embodiments P1-P92, wherein the CFH polypeptide or biologically active fragment and/or variant thereof is administered intravitreally.

Embodiment P95: The method of any one of embodiments P1-P67, wherein the subject is homozygous for at least one of the one or more CFH mutations.

Embodiment P96: The method of any one of embodiments P1-P67, wherein the subject is heterozygous for at least one of the one or more CFH mutations.

Embodiment P97: The method of any one of embodiments P1-P95, wherein the subject is homozygous for a Y402H mutation.

Embodiment P98: The method of any one of embodiments P1-P95, wherein the subject is a subject in whom it has been determined is homozygous for a Y402H mutation.

1. A method of treating a subject having a disease or disorder associated with undesired activity of the alternative complement pathway, comprising the step of administering to the subject a complement factor H (CFH) polypeptide or biologically active fragment and/or variant thereof, wherein the subject has one or more CFH, complement component 3 (C3), and/or complement factor B (CFB) gene mutations.

2. A method of treating a subject having age-related macular degeneration (AMD), comprising the step of administering to the subject a complement factor H (CFH) polypeptide or biologically active fragment and/or variant thereof, wherein the subject has one or more CFH, C3, and/or CFB gene mutations.

3. A method of treating a subject having an ocular disease associated with neovascularization, the method comprising the steps of:
(a) administering to the subject a complement factor H (CFH) polypeptide or biologically active fragment and/or variant thereof; and
(b) administering to the subject a VEGF antagonist,
optionally wherein the subject has one or more CFH, C3, and/or CFB gene mutations.

4. The method of claim 3, wherein the VEGF antagonist comprises an antibody or antigen-binding fragment thereof that binds VEGF.

5. The method of claim 4, wherein the antibody or antigen-binding fragment thereof is selected from the group consisting of ranibizumab and bevacizumab.

6. The method of claim 3, wherein the VEGF antagonist is aflibercept.

7. The method of any one of claims 3-6, wherein the ocular disease associated with neovascularization is neovascular AMD.

8. The method of any one of claims 3-6, wherein the ocular disease associated with neovascularization is diabetic retinopathy.

9. The method of claim 8, wherein the ocular disease associated with neovascularization is diabetic macular edema.

10. The method of any one of claims 1-9, wherein the CFH polypeptide or biologically active fragment and/or variant thereof is capable of acting as a cofactor with CFI to facilitate C3b cleavage.

11. The method of any one of claims 1-10, wherein the CFH polypeptide or biologically active fragment and/or variant thereof is capable of diffusing across the Bruch's membrane.

12. The method of any one of claims 1-11, wherein the CFH polypeptide or biologically active fragment and/or variant thereof is capable of binding C3b.

13. The method of any one of claims 1-12, wherein the CFH polypeptide or biologically active fragment and/or variant thereof is capable of facilitating the breakdown of C3b.

14. The method of any one of claims 1-13, wherein the CFH polypeptide or biologically active fragment and/or variant thereof is capable of binding to a cell surface.

15. The method of any one of claims 1-14, wherein the CFH polypeptide or biologically active fragment and/or variant thereof is capable of binding to heparin.

16. The method of any one of claims 1-15, wherein the CFH polypeptide or biologically active fragment and/or variant thereof is capable of reducing C5b9 levels generated as a result of complement activation.

17. The method of any one of claims 1-16, wherein the CFH polypeptide or biologically active fragment and/or variant thereof is capable of inhibiting hemolysis.

18. The method of any one of claims 1-17, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 3, or a biologically active fragment thereof.

19. The method of any one of claims 1-17, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 1, or a biologically active fragment thereof.

20. The method of any one of claims 1-17, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 2, or a biologically active fragment thereof.

21. The method of any one of claims 1-17, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 4, or a biologically active fragment thereof.

22. The method of any one of claims 1-17, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 5, or a biologically active fragment thereof.

23. The method of any one of claims 1-17, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 6, or a biologically active fragment thereof.

24. The method of any one of claims 1-17, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 7, or a biologically active fragment thereof.

25. The method of any one of claims 1-17, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 8, or a biologically active fragment thereof.

26. The method of any one of claims 1-17, wherein the CFH polypeptide or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 9, or a biologically active fragment thereof.

27. The method of any one of claims 1-26, wherein the CFH polypeptide or biologically active fragment and/or variant thereof is encoded by a nucleotide sequence that is at least 70%, 75%, 80%, 85%, 90%, 91%, 92%, 93%, 94%, 95%, 96%, 97%, 98%, 99% or 100% identical to the nucleotide sequence of any one of SEQ ID NOs: 10-13, or a biologically active fragment thereof.

28. The method of any one of claims 1-27, wherein the CFH polypeptide or biologically active fragment and/or variant thereof comprises the V62 polymorphism.

29. The method of any one of claims 1-28, wherein the CFH polypeptide or biologically active fragment and/or variant thereof comprises the Y402 polymorphism.

30. The method of any one of claims 1-29, wherein the CFH polypeptide or biologically active fragment and/or variant thereof is a mature CFH polypeptide or biologically active fragment and/or variant thereof.

31. The method of any one of claims 1-30, wherein the subject is a human.

32. The method of claim 31, wherein the human is at least 40 years of age.

33. The method of claim 31, wherein the human is at least 50 years of age.

34. The method of claim 31, wherein the human is at least 65 years of age.

35. The method of any one of claims 1-34, wherein the CFH polypeptide or biologically active fragment and/or variant thereof is administered locally.

36. The method of claim 35, wherein the CFH polypeptide or biologically active fragment and/or variant thereof is administered intravitreally.

37. The method of any one of claims 1-34, wherein the CFH polypeptide or biologically active fragment and/or variant thereof is administered systemically.

38. The method of any one of claims 1-37, wherein the subject has a mutation in the subject's CFH gene, optionally wherein the mutation is a loss-of-function mutation.

39. The method of claim 38, wherein the subject has one or more of the following CFH mutations: Y402H, R2T, L3V, R53C, R53H, S58A, D90G, D130N, R175Q, R175P, I221V, R303W, R303Q, Q400K, P503A, R567G, G650V, S8901, T956M, G1194D, and R1210C.

40. The method of claim 38, wherein the subject has one or more of the following CFH mutations: R2T, R53C, R53H, S58A, D130N, R175Q, R175P, I221V, R303W, R303Q, P503A, R567G, G650V, G1194D, and R1210C.

41. The method of claim 38 or 39, wherein the subject has a Y402H mutation.

42. The method of claim 41, wherein the subject is homozygous for a Y402H mutation.

43. The method of any one of claims 38-40, wherein the subject has an R2T mutation.

44. The method of claim 38 or 39, wherein the subject has an L3V mutation.

45. The method of any one of claims 38-40, wherein the subject has an R53C mutation.

46. The method of any one of claims 38-40, wherein the subject has an R53H mutation.

47. The method of any one of claims 38-40, wherein the subject has an S58A mutation.

48. The method of claim 38 or 39, wherein the subject has a D90G mutation.

49. The method of any one of claims 38-40, wherein the subject has a D130N mutation.

50. The method of any one of claims 38-40, wherein the subject has an R175Q mutation.

51. The method of any one of claims 38-40, wherein the subject has an R175P mutation.

52. The method of any one of claims 38-40, wherein the subject has an I221V mutation.

53. The method of any one of claims 38-40, wherein the subject has an R303W mutation.

54. The method of any one of claims 38-40, wherein the subject has an R303Q mutation.

55. The method of claim 38 or 39, wherein the subject has a Q400K mutation.

56. The method of any one of claims 38-40, wherein the subject has a P503A mutation.

57. The method of any one of claims 38-40, wherein the subject has an R567G mutation.

58. The method of any one of claims 38-40, wherein the subject has a G650V mutation.

59. The method of any one of claims 38-39, wherein the subject has an S890I mutation.

60. The method of any one of claims 38-40, wherein the subject has a T956M mutation.

61. The method of any one of claims 38-40, wherein the subject has a G1194D mutation.

62. The method of any one of claims 38-40, wherein the subject has an R1210C mutation.

63. The method of any one of claims 38-62, wherein the subject expresses a mutant CFH polypeptide having reduced CFH activity as compared to a wildtype CFH polypeptide.

64. The method of claim 63, wherein the CFH activity is the ability to bind to C3b.

65. The method of claim 63, wherein the CFH activity has the ability to act as a cofactor with CFI and facilitate C3b cleavage.

66. The method of claim 63, wherein the CFH activity is the ability to bind to a cell surface.

67. The method of claim 63, wherein the CFH activity is the ability to bind to heparin.

68. The method of claim 63, wherein the CFH activity is the ability to reduce C5b9 levels generated as a result of complement activation.

69. The method of claim 63, wherein the CFH activity is the ability to inhibit hemolysis.

70. The method of any one of claims 63-69, wherein the wildtype CFH polypeptide comprises the amino acid sequence of SEQ ID NO: 1, 2, or 3.

71. The method of any one of claims 1-70, wherein the subject has a mutation in the subject's C3 gene, optionally wherein the mutation is a gain-of-function mutation.

72. The method of claim 71, wherein the subject has one or more of the following C3 mutations: R102G, K155Q, V619M, and R735W.

73. The method of any one of claims 1-72, wherein the subject has a mutation in the subject's CFB gene, optionally wherein the mutation is a gain-of-function mutation.

74. The method of claim 73, wherein the subject has the I242L mutation of CFB.

75. The method of any one of claims 1-74, wherein the subject is homozygous for CFH 62V, C3 102G, and complement factor B (CFB) 32R.

76. The method of any one of claims 1-75, wherein the subject is homozygous for at least one of the one or more CFH, C3, and/or CFB mutations.

77. The method of any one of claims 1-76, wherein the subject is heterozygous for at least one of the one or more CFH, C3, and/or CFB mutations.

78. The method of any one of claims 1-77, wherein the subject has been determined to have the one or more CFH, C3, and/or CFB mutations.

79. The method of any one of claims 1-78, wherein the subject has atypical hemolytic uremic syndrome (aHUS).

80. The method of any one of claims 1-79, wherein the subject has a renal disease or complication.
